(19) United States
(12) Patent Application Publication
(10) Pub. No.: US 2013/0315955 A1
(43) Pub. Date: Nov. 28, 2013

(54) STABILITY AND POTENCY OF HEMAGGLUTININ

(71) Applicants: Kathleen Holtz, Cheshire, CT (US); Erin Matthews, Wallingford, CT (US); David Rhodes, Storrs, CT (US); Indresh Srivastava, Meriden, CT (US)

(72) Inventors: Kathleen Holtz, Cheshire, CT (US); Erin Matthews, Wallingford, CT (US); David Rhodes, Storrs, CT (US); Indresh Srivastava, Meriden, CT (US)

(73) Assignee: Protein Sciences Corporation, Meriden, CT (US)

(21) Appl. No.: 13/838,796

(22) Filed: Mar. 15, 2013

Related U.S. Application Data
(60) Provisional application No. 61/624,222, filed on Apr. 13, 2012.

(51) Int. Cl. A61K 39/145

(52) U.S. Cl. CPC A61K 39/145
USPC 424/210.1; 530/396; 435/320.1; 435/23

(57) ABSTRACT

The present invention relates to methods of improving the stability and maintaining the potency of recombinant hemagglutinin formulations, in particular, recombinant influenza hemagglutinin (rHA). In particular, Applicants have shown that the stability of rHA formulations may be significantly improved by mutating cysteine residues or by formulating with a reducing agent and sodium citrate.

### INCORPORATION BY REFERENCE

### Related Applications and Incorporation by Reference

This application claims benefit of U.S. provisional patent application Ser. No. 61/624,222 filed Apr. 13, 2012.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was supported, in part, by BARDA grant number: HHSO100200900106C. The federal government may have certain rights to this invention.

### FIELD OF THE INVENTION

The present invention relates to methods of improving the stability and maintaining the potency of recombinant hemagglutinin formulations, in particular, recombinant influenza hemagglutinin (rHA).

### BACKGROUND OF THE INVENTION

Epidemic influenza occurs annually and is a cause of significant morbidity and mortality worldwide. Children have the highest attack rate, and are largely responsible for transmission of influenza viruses in the community. The elderly and persons with underlying health problems are at increased risk for complications and hospitalization from influenza infection.

Influenza viruses are highly pleomorphic particles composed of two surface glycoproteins, hemagglutinin (HA) and neuraminidase (NA). The HA mediates attachment of the virus to the host cell and viral-cell membrane fusion during penetration of the virus into the cell. The influenza virus genome consists of eight single-stranded negative-sense RNA segments of which the fourth largest segment encodes the HA gene. The influenza viruses are divided into types A, B and C based on antigenic differences. Influenza A viruses are described by a nomenclature which includes the sub-type or type, geographic origin, strain number, and year of isolation, for example, A/Beijing/353/89. There are at least 13 sub-types of HA (H1-H13) and 9 subtypes of NA (N1-N9). All subtypes are found in birds, but only H1-H3 and N1-N2 are found in humans, swine and horses (Murphy and Webster, “Orthomyxoviruses”, in Virology, ed. Fields, B. N., Knipe, D. M., Chanock, R. M., 1091-1152 (Raven Press, New York, (1990)).

Antibodies to HA neutralize the virus and form the basis for natural immunity to infection by influenza (Clements, “Influenza Vaccines”, in Vaccines: New Approaches to Immunological Problems, ed. Ronald W. Ellis, pp. 129-150 (Butterworth-Heinemann, Stoneham, Mass. 1992)). Antigenic variation in the HA molecule is responsible for frequent outbreaks to influenza and for limited control of infection by immunization.

The three-dimensional structure of HA and the interaction with its cellular receptor, sialic acid, has been extensively studied (Wilson, et al, “Structure of the hemagglutinin membrane glycoprotein of influenza virus at 3A.degree. resolution” Nature 289:366-378 (1981); Weis, et al, “Structure of the influenza virus hemagglutinin complexed with its receptor, sialic acid” Nature, 333:426-431 (1988); Murphy and Webster, 1990). The HA molecule is present in the virion as a trimer. Each HA monomer (HA0) exists as two chains, HA1 and HA2, linked by a single disulfide bond. Infected host cells produce a precursor glycosylated polypeptide (HA0) with a molecular weight of about 85,000 Da, which in vivo, is subsequently cleaved into HA1 and HA2.

The presence of influenza HA-specific neutralizing IgG and IgA antibody is associated with resistance to infection and illness (Clements, 1992). Inactivated whole virus or partially purified (split subunit) influenza vaccines are standardized to the quantity of HA from each strain. Influenza vaccines usually include 7 to 25 micrograms HA from each of three strains of influenza.

Most licensed influenza vaccines consist of formalin-inactivated whole or chemically split subunit preparations from two influenza A subtype (H1N1 and H3N2) and one influenza B subtype viruses. Prior to each influenza season, the U.S. Food and Drug Administration's Vaccines and Related Biological Products Advisory Committee recommends the composition of a trivalent influenza vaccine for the upcoming season. Vaccination of high-risk persons each year before the influenza season is the most effective measure for reducing the impact of influenza. Limitations of the currently available vaccines include low use rates; poor efficacy in the elderly and in young children; production in eggs (especially for those allergic to egg proteins); antigenic variation; and adverse reactions.

Seed viruses for influenza A and B vaccines are naturally occurring strains that accumulate to high titers in the allantoic fluid of chicken eggs. Alternatively, the strain for the influenza A component is a reassortant virus with the correct surface antigen genes. A reassortant virus is one that, due to segmentation of the viral genome, has characteristics of each parental strain. When more than one influenza viral strains infect a cell, these viral segments mix to create progeny virion containing various assortments of genes from both parents.

Protection with whole or split influenza vaccines is short-lived and wanes as antigenic drift occurs in epidemic strains of influenza. Influenza viruses undergo antigenic drift as a result of immune selection of viruses with amino acid sequence changes in the hemagglutinin molecule. Ideally, the vaccine strains match the influenza virus strains causing disease. The current manufacturing process for influenza vaccines, however, is limited by propagation of the virus. For example, not all influenza virus strains replicate well in eggs or mammalian cells; thus the viruses must be adapted or viral reassortants constructed. Extensive heterogeneity occurs in the hemagglutinin of egg-grown influenza viruses as compared to primary isolates from infected individuals grown in mammalian cells (Wang, et al, Virol. 171:275-279 (1989); Rajakumar, et al, Proc. Natl. Acad. Sci. USA 87:4154-4158 (1990)). The changes in HA during the selection and manufacture of influenza vaccines can result in a mixture of antigenically distinct subpopulations of virus. The viruses in the vaccine may therefore differ from the variants within the epidemic strains, resulting in suboptimal levels of protection.

Recombinant hemagglutinin (rHA) based influenza vaccine Flublok™ (see, e.g., U.S. Pat. No. 5,762,939) was recently approved in the US as an alternative to the traditional egg-derived flu vaccines. rHA from multiple strains of the virus were expressed in baculovirus, purified, characterized and stored at 2-8° C. before final formulation. However, an initial loss of potency is usually observed. This loss of potency is typically greater for H3 rHA proteins compared to other rHA proteins.

There is a need for alternative flu vaccines that have greater stability, that is, vaccines that retain potency for longer periods of time.

Citation or identification of any document in this application is not an admission that such document is available as prior art to the present invention.

### SUMMARY OF THE INVENTION

The present invention relates to isolated, non-naturally occurring recombinant hemagglutinin (rHA) proteins which may comprise one or more cysteine mutations. The cysteine mutation(s) may be in the carboxy terminus region of the rHA protein which may include the transmembrane (TM) and cytosolic domain (CT).

The present invention is based, in part, on Applicants' finding that the stability of HA is decreased by disulfide cross linking and that this appears to be the primary mechanism of potency loss. There are two methods of addressing this issue—mutagenesis to remove the cysteine residues involved in the cross linking or formulation to inhibit the cross linking reaction.

In particular, Applicants have demonstrated that mutations in the H3 protein increase its stability and maintain potency longer. The present invention relates to isolated, non-naturally occurring recombinant hemagglutinin (rHA) proteins which may comprise one or more cysteine mutations. The cysteine mutation(s) may be in the carboxy terminus region of the rHA protein which may include the transmembrane (TM) and cytosolic domain (CT). Without being bound by any limitations, it is believed that the mutations do not disrupt trimer formation which may be critical for immunogenicity and efficacy. In addition, Applicants have demonstrated that a formulation approach involving a reducing agent and an antioxidant is capable of significantly improving the shelf life of HA.

The rHA protein may be any H3 protein. The H3 protein may be isolated from a Victoria, Perth, Brisbane, or Wisconsin strain. The Victoria strain may be a Victoria/361/2011 strain. The Perth strain may be a Perth/16/2009. The Brisbane strain may be a Brisbane/16/2007 strain and the Wisconsin strain may be a A/Wisconsin/67/05 strain.

The rHA protein may be any H1 protein. The H1 protein may be isolated from a California or Solomon strain. The California strain may be a California/07/2009 strain and the Solomon strain may be a Solomon Is/03/2006 strain.

In another embodiment, the rHA protein may be any H2, H5, H7 and/or H9 protein.

The rHA protein may be any B protein. The B protein may be isolated from a Brisbane, Florida, Ohio, Jiangsu or Hong Kong strain. The Brisbane strain may be a Brisbane/60/2008 strain. The Florida strain may be a Florida/04/2006 strain, the Ohio strain may be a Ohio/01/2005 strain, the Jiangsu strain may be a Jiangsu/10/2003 strain and the Hong Kong strain may be a Hong Kong/330/2001 strain.

The present invention encompasses any HA protein with transmembrane or cytosolic cysteine residues that are mutated to non-cysteine residues to increase the stability and/or potency of the HA antigen(s) in an influenza vaccine. The present invention also encompasses the encoding and expression of nucleotide sequences for any of the proteins disclosed herein. Advantageously, the vector may be a baculovirus vector. The present invention also relates to an influenza vaccine which may comprise any of the proteins disclosed herein and/or a baculovirus vector encoding and expressing a nucleotide sequence expressing any of the proteins disclosed herein.

The present invention also relates to methods for stabilizing protein vaccines which may comprise adding an antioxidant and a low toxicity reducing agent and formulations thereof. In one embodiment, the antioxidant may be citrate. The concentration of the antioxidant may be at least about 5 mg/ml, at least about 10 mg/ml or at least about 20 mg/ml. In another embodiment, the reducing agent may be a thioglycolate, such as sodium thioglycolate or a thioglycerol, such as monothioglycerol. The concentration of the reducing agent may be about 0.2 mg/ml.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises,” “comprised,” “comprising,” and the like can have the meaning attributed to it in U.S. Patent law; e.g., they can mean “includes,” “included,” “including,” and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1C. The table denotes a representative HA sequence for H3 Perth and all the possible symmetrical orientations the amino acids residue could occur in a trimer configuration. The drawings depict a trimer configuration with 7 positions labeled A through G on the left and one possible orientation on the right. Note in the illustration on the right, 3 of the 5 cysteines occur in the interface while two are available for disulfide bonding to other trimers.

FIG. 2. Sequence Alignment of Hemagglutinin Proteins Derived from H1, B and H3 Human Influenza Strains. Shown below is a sequence alignment of the transmembrane (TM) and cytoplasmic tail (CT) domains of hemagglutinin proteins. The cysteine residues are highlighted in yellow.

FIG. 3. Average Stability Trends for Recombinant Hemagglutinins Manufactured Between 2007-2011 According to Subtype: B, H1, and H3, and the 2010 Stability Profile for H3/Perth rHA. Shown is a graph of relative potency as a function of time according to subtype for manufacturing batches produced between 2007 and 2011, and for batches of H3/Perth manufactured in the 2010 campaign. The relative potency data for one to three batches of rHA produced in each manufacturing campaign between 2007 through 2011 were used to generate the trend lines for each subtype. The subtypes represent multiple rHA proteins derived from different influenza strains.

FIG. 4. Purity of H3 rHA proteins. The purified H3 rHA proteins have a purity of 100% by reducing SDS-PAGE gel analysis using a 1 μg/lane loading. The study criterion for purity by SDS-PAGE is ≧85%.

FIG. 5. Wild-type H3 rHA and the Cys mutants are resistant to trypsin indicating that the rHA proteins are properly folded and trimeric. All H3 rHAs met the study criteria for the assay, visible bands for HA1 and HA2.

FIG. 6. Potency by SRID After 1 month at 25° C., the wild-type H3 rHA protein showed the greatest potency drop and stabilized at a relative potency of ˜40%. The relative potency for the 5Cys H3 rHA stabilized at ˜60%. The potency drop for the 3Cys H3 rHA was less than 20%, and the 2Cys H3 rHA shows no potency loss. All three Cys H3 rHA variants meet study requirements for relative potency (RP) on day 28. Study criteria: 28-Day RP_{mutant rHA}≧28-Day RP_{wild-type rHA}

FIG. 7A. Non-reducing and reducing SDS-PAGE profiles on days 0, 7, 14 and 28 for the wild-type H3 rHA protein and the Cys mutant rHAs.

FIG. 7B. The non-reducing SDS-PAGE gels of FIG. 7A were analyzed using Carestream's Molecular Imaging Software. The intensity profiles from the imaging analysis are shown for day 0 of the study

FIG. 7C, Densitometry was performed on the non-reducing SDS-PAGE gels at each time point and for each H3 rHA protein. The band intensities for the monomeric rHA protein (HA0) and the higher cross-linked forms of the rHA protein (aggregation) were determined. A ratio of the aggregates and HA0 is presented.

FIG. 8. The RP-HPLC profiles for the 3Cys and 2Cys mutants are comparable but different from the wild-type and 5Cys mutant. The 3Cys and 2Cys rHA are largely un-cross-linked and elute as a single peak while the wild-type and 5Cys rHA elute in multiple peaks due to various cross-linked populations of protein. Populations of cross-linked rHA are retained on the column due to increased hydrophobicity and elute later.

FIG. 9. Size exclusion chromatography (SEC) analysis of WT and mutant rHAs. By SEC, the retention time for all H3 rHA proteins elute is the same retention time. Extrapolated molecular weights in the range of 2.4-2.6 MDa were observed for the WT and mutant H3 rHA proteins. Using an approximate MW for the monomer of ˜70 kDa. the number of monomers per particle/rosette is estimated to be 35-38.

FIG. 10. Representative electron microscopy (EM) images of the wild-type H3 rHA and the three cysteine mutant rHA proteins. All images are of 135,000× magnification of the respective rHA proteins. The black bar represents 100 nm. The rHA protein samples were stored at 25° C. for approximately 2 months prior to EM analysis. Similar rosette sizes and density are observed for the wild-type and mutant H3 rHA proteins.

FIG. 11. Thermal denaturation curves for the H3 rHA wild-type and cysteine mutants using differential scanning fluorimetry (DSF). The melting temperature (Tm) is measured by an increase in the fluorescence of a dye with affinity for hydrophobic parts of the protein that become exposed as it unfolds. The fluorescence intensity is plotted as a function of temperature for all rHA proteins (A) and the transition point is more clearly observed in the second derivative plots (B). Representative second derivative thermal denaturation curves for each rHA and corresponding Tm values are shown in plots C-F.

FIG. 12. Hemagglutination Inhibition (HI) assay using rabbit anti-H3 rHA antiserum and sheep anti-H3 HA antiserum and the wild-type and cysteine mutant H3 rHA proteins. rHA proteins were standardized to have 4 HA units/25 μL which results in agglutination in the first four wells of the back titration (BT). The BT endpoint is denoted by a solid gray line in between rows D and E. The standardized quantity of each rHA was mixed with serially diluted rabbit and sheep antiserum in the columns labeled Ab. The HI endpoint is denoted by a dashed gray line in Ab columns. The dilution of antiserum that completely inhibits hemagglutination is the HI titer.

FIG. 13. Free Thiol and Free Cys-549 (Peptide Mapping) Results for H3 rHA. Shown on the left-hand side is the change in the free thiol content on an absolute scale (top) and relative to day 0 (bottom) for different formulations of H3 rHA over a 28 day study. Shown on the right-hand side is the loss of free cysteine at position 549 for different formulation and storage condition in a 28 day stability study.

FIG. 14. Relative Potency Loss and Relative Free-Thiol Loss for H3 rHA. The potency loss and the free thiol loss relative to their day 0 values are plotted for different formulations of H3 rHA.

FIG. 15. Relative Potency Loss and Relative Free Cys549 Loss for H3 rHA. The potency loss and the free Cys549 loss relative to their day 0 values are plotted for different formulations of H3 rHA.

FIG. 16 depicts H1/Brisbane SRID Potency. Left panels are raw potency data (±SD) and right panels are potency relative to day 0.

FIG. 17 depicts H3/Brisbane SRID Potency. Left panels are raw potency data (±SD) and right panels are potency relative to day 0.

FIG. 18 depicts B/Brisbane SRID Potency. Left panels are raw potency data (+SD) and right panels are potency relative to day 0.

FIG. 19 depicts Day-0 potency data.

FIG. 20 depicts potency loss under accelerated conditions. Potency loss (%/day) was calculated from linear fits of relative potency data (percentage of day 0 potency as a function of time) for 21 days. Thus, low values represent better stability and high values represent rapid loss of potency. Upper panels were from samples stored at 35° C. and lower panels from samples stored at 25° C.

FIG. 21 depicts SDS-PAGE results.

FIG. 22 depicts potency data—The left panels show potency (μg/mL) and the right panels show these results plotted relative to the day-0 potency. The traces are: control—0.035% Triton X-100, Triton X-100 concentrations of 0.05%. 0.1%, and 0.2%, and STG-Citrate.

FIGS. 23A-B depict SDS-PAGE results—Gels are shown from day 0 (FIG. 23A) and day 14 (FIG. 23B). In each gel, non-reducing and reducing conditions were run for control (0.035% Triton X-100), 0.05% Triton X-100 (T05), 0.1% Triton X-100 (T10), 0.2% Triton X-100 (T20), and the STG-citrate formulation. The numbers at left are molecular weights of standard proteins and numbers at right indicate the size of cross-linked oligomers: HA0 (monomer), dimer, trimer, etc.

FIG. 24 depicts DLS results—The results for control and 0.2% Triton X-100 are shown for days 0, 7, and 14.

FIG. 25 is a plot of HAI titer results, plotted on a log₁₀ scale. The horizontal bars indicate titer results for individual mice and the circles indicate the mean titer calculated from all eight mice in each group. Note that some of the bars represent more than one mouse; for example, in the low dose Control, three mice had titers of 80 and three had titers of 40.

FIG. 26 is a scatter plot of HAI and ELISA results. Results from each method are plotted to compare the results in each test animal. The points were fit to a straight line and the resulting equation and R² are shown.

### DETAILED DESCRIPTION OF THE INVENTION

The present invention may be applied generally to protein vaccines. Advantageously, the protein vaccine is an influenza vaccine. The influenza vaccine may comprise hemagglutinin formulations, advantageously recombinant hemagglutinin formulations, in particular, recombinant influenza hemagglutinin (rHA). In a particularly advantageous embodiment, the influenza vaccine may be a monovalent, divalent, trivalent or quadrivalent vaccine. The vaccines of U.S. Pat. Nos. 5,762,939 or 6,245,532 with the herein disclosed cysteine mutations are contemplated. In one advantageous embodiment, the vaccine may comprise a recombinant rHA with one or more cysteine substitutions and/or mutations.

The hemagglutinin (HA) molecule contains many cysteine amino acids. Applicant's invention concerns, in part, the cysteines in the transmembrane and cytoplasmic regions of the hemagglutinin molecules located in the carboxy terminus.

The transmembrane region of HA is expected to form an alpha helix in continuation with the extracellular helix. Cysteines found in alpha helical transmembrane domains (domains spanning the membrane bilayer) are unlikely to spontaneously engage in covalent disulfide bonds, as the membrane bilayer is a non-oxidizing environment [Matthews, E. E., et al., Thrombopoietin receptor activation: transmembrane helix dimerization, rotation, and allosteric modulation. FASEB J, 2011. 25(7): p. 2234-44]. Likewise, intracellular cysteines are exposed to the reducing environment inside the cell. Additionally, the 3 C-terminal cysteines may be pamitoylated [Kordyukova, L. V., et al., S acylation of the hemagglutinin of influenza viruses: mass spectrometry reveals site-specific attachment of stearic acid to a transmembrane cysteine. J Virol, 2008. 82(18): p. 9288-92, Kordyukova, L. V., et al., Site-specific attachment of palmitate or stearate to cytoplasmic versus transmembrane cysteines is a common feature of viral spike proteins. Virology, 2010. 398(1): p. 49-56 and Serebryakova, M. V., et al., Mass spectrometric sequencing and acylation character analysis of C-terminal anchoring segment from Influenza A hemagglutinin. Eur J Mass Spectrom (Chichester, Eng), 2006. 12(1): p. 51-62]. Thus, in their native folded state, transmembrane and intracellular cysteines in the influenza HA are expected to exhibit a low level of disulfide crosslinking. However, in the process of expression and purification of HA, these cysteines may be exposed to a chemical environment that promotes disulfide crosslinking.

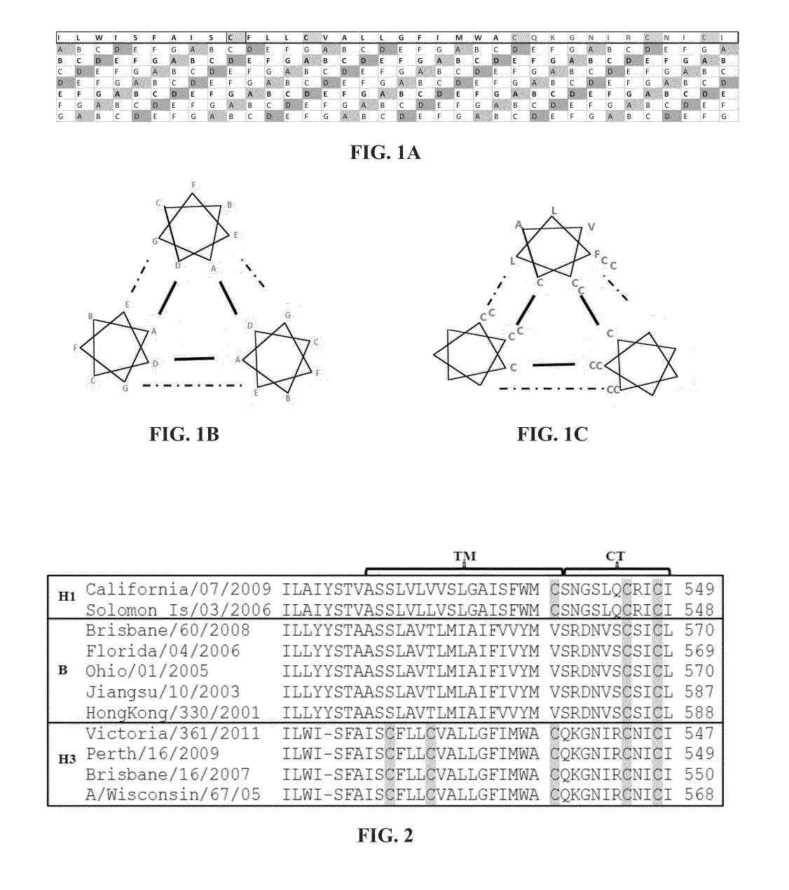

Regardless of the exact primary sequence of the protein, the transmembrane region of HA molecules are expected to form alpha helices that pack in at least a trimeric fold (higher order oligomers are also present both in the native protein and in Applicant's vaccine) [Markovic. I., et al., Synchronized activation and refolding of influenza hemagglutinin in multimeric fusion machines. J Cell Biol, 2001. 155(5): p. 833-44]. The alpha helical, membrane spanning region may be defined with algorithms such as those used in the program TMHMM [Krogh, A., et al., Predicting transmembrane protein topology with a hidden Markov model: application to complete genomes. J Mol Biol, 2001. 305(3): p. 567-80, Sonnhammer, E. L., G. von Heijne, and A. Krogh, A hidden Markov model for predicting transmembrane helices in protein sequences. Proc Int Conf Intell Syst Mol Biol, 1998. 6: p. 175-82]. The intracellular portion may extend the alpha helix. FIG. 1 shows a representative HA sequence from H3 Perth and the 7 possible symmetrical alpha helical trimer configurations with interfacial positions highlighted in pink (A and D). Amino acids with a spacing of 3 or 4 may be found on the same face of an alpha helix and cysteines in those positions can form disulfide bonds between two adjacent helices, thus covalently linking helices. Cysteines on the outside of the helices may participate in the covalent crosslinking of higher order oligomers. Since modifications of the transmembrane and cytoplasmic domains of HA are known to affect the entire structure of HA [Kozerski, C., et al., Modification of the cytoplasmic domain of influenza virus hemagglutinin affects enlargement of the fusion pore. J Virol, 2000. 74(16): p. 7529-37, Melikyan, G. B., et al., Amino acid sequence requirements of the transmembrane and cytoplasmic domains of influenza virus hemagglutinin for viable membrane fusion. Mol Biol Cell, 1999. 10(6): p. 1821-36 and Melikyan, G. B., et al., A point mutation in the transmembrane domain of the hemagglutinin of influenza virus stabilizes a hemifusion intermediate that can transit to fusion. Mol Biol Cell, 2000. 11(11): p. 3765-75], Applicants propose that disulfide crosslinking may be altering the overall stability and structure of the HA molecule and thus potency of the HA vaccine. This unique (non-biological) environment occurring during the manufacture of HA vaccine is allowing non-native crosslinking to occur and Applicants' discovery presented here overcomes the constraints of that environment during the manufacturing and storage of HA vaccine.

Therefore, the present invention encompasses, in part, a method of stabilizing a rHA protein which may comprise identifying one or more cysteine residues in the rHA protein, mutating the one or more cysteine residues to an amino acid residue that is not cysteine and does not disrupt trimer formation, thereby stabilizing the rHA protein. Identifying and mutating a cysteine residue and verifying that the resultant mutation does not disrupt trimer formation is well known to one of skill in the art. The resultant mutant protein may also be tested for immunogenicity and efficacy.

In one advantageous embodiment, the present invention relates to methods for stabilizing protein vaccines which may comprise adding an antioxidant and a low toxicity reducing agent.

In another advantageous embodiment, the vaccine may comprise a recombinant vector containing and expressing a rHA with one or more cysteine mutations. In a particularly advantageous embodiment, the recombinant vector may be a baculovirus vector.

Baculoviruses are DNA viruses in the family Baculoviridae. These viruses are known to have a narrow host-range that is limited primarily to Lepidopteran species of insects (butterflies and moths). The baculovirus Autographa californica Nuclear Polyhedrosis Virus (AcMNPV), which has become the prototype baculovirus, replicates efficiently in susceptible cultured insect cells. AcMNPV has a double-stranded closed circular DNA genome of about 130,000 base-pairs and is well-characterized with regard to host range, molecular biology, and genetics.

Many baculoviruses, including AcMNPV, form large protein crystalline occlusions within the nucleus of infected cells. A single polypeptide, referred to as a polyhedrin, accounts for approximately 95% of the protein mass of these occlusion bodies. The gene for polyhedrin is present as a single copy in the AcMNPV viral genome. Because the polyhedrin gene is not essential for virus replication in cultured cells, it can be readily modified to express foreign genes. The foreign gene sequence is inserted into the AcMNPV gene just 3′ to the polyhedrin promoter sequence such that it is under the transcriptional control of the polyhedrin promoter.

Recombinant baculoviruses that express foreign genes are constructed by way of homologous recombination between baculovirus DNA and chimeric plasmids containing the gene sequence of interest. Recombinant viruses can be detected by virtue of their distinct plaque morphology and plaque-purified to homogeneity.

Baculoviruses are particularly well-suited for use as eukaryotic cloning and expression vectors. They are generally safe by virtue of their narrow host range which is restricted to arthropods. The U.S. Environmental Protection Agency (EPA), has approved the use of three baculovirus species for the control of insect pests. AcMNPV has been applied to crops for many years under EPA Experimental Use Permits.

In an advantageous embodiment, a wild type baculovirus is the vector, such as the insect baculovirus Autographa californica nuclear polyhedrosis virus (AcMNPV) (Li J A, Happ B, Schetter C, Oellig C, Hauser C, Kuroda K, Knebel-Mörsdorf D, Klenk H D, Doerfler W. The expression of the Autographa californica nuclear polyhedrosis virus genome in insect cells. Vet Microbiol. 1990 June; 23(1-4):73-8).

The baculovirus vectors of U.S. Pat. Nos. 7,964,767; 7,955,793; 7,927,831; 7,527,967; 7,521,219; 7,416,890; 7,413,732; 7,393,524; 7,329,509; 7,303,882; 7,285,274; 7,261,886; 7,223,560; 7,192,933; 7,101,966; 7,070,978; 7,018,628; 6,852,507; 6,814,963; 6,806,064; 6,555,346; 6,511,832; 6,485,937; 6,472,175; 6,461,863; 6,428,960; 6,420,523; 6,403,375; 6,368,825; 6,342,216; 6,338,846; 6,326,183; 6,310,273; 6,284,455; 6,261,805; 6,245,528; 6,225,060; 6,190,862; 6,183,987; 6,168,932; 6,126,944; 6,096,304; 6,090,584; 6,087,165; 6,057,143; 6,042,843; 6,013,433; 5,985,269; 5,965,393; 5,939,285; 5,919,445; 5,891,676; 5,871,986; 5,869,336; 5,861,279; 5,858,368; 5,843,733; 5,840,541; 5,827,696; 5,824,535; 5,789,152; 5,762,939; 5,753,220; 5,750,383; 5,686,305; 5,665,349; 5,641,649; 5,639,454; 5,605,827; 5,605,792; 5,583,023; 5,571,709; 5,521,299; 5,516,657; 5,322,774; 5,290,686; 5,244,805; 5,229,293; 5,194,376; 5,186,933; 5,169,784; 5,162,222; 5,147,788; 5,110,729; 5,091,179; 5,077,214; 5,071,748; 5,011,685; 4,973,667; 4,879,236; 4,870,023 or 4,745,051 may also be contemplated for the present invention.

In another embodiment, the vector may further comprise a globin terminator (see, e.g., Mapendano C K Mol. Cell. 2010 Nov. 12; 40(3):410-22, Brennan S O Hemoglobin. 2010; 34(4):402-5, Haywood A Ann Hematol. 2010 December; 89(12):1215-21. Epub 2010 Jun. 22, Banerjee A PLoS One. 2009 Jul. 9; 4(7):e6193, West S Mol. Cell. 2009 Feb. 13; 33(3):354-64, Eberle A B Nat Struct Mol. Biol. 2009 January; 16(1):49-55. Epub 2008 Dec. 7, West S Mol. Cell. 2008 Mar. 14; 29(5):600-10, Tsang J C Clin Chem. 2007 December; 53(12):2205-9. Epub 2007 Oct. 19, Yingzhong Y Gene. 2007 Nov. 15; 403(1-2):118-24. Epub 2007 Aug. 22, Foulon K Hemoglobin. 2007; 31(1):31-7, Frischknecht H Haematologica. 2007 March; 92(3):423-4. Review, Wang J J Am Chem. Soc. 2006 Jul. 12; 128(27):8738-9, Gromak N Mol Cell Biol. 2006 May; 26(10):3986-96, West S RNA. 2006 April; 12(4):655-65, Chan A Y Clin Chem. 2006 March; 52(3):536-7, Mo Q H J Clin Pathol. 2005 September; 58(9):923-6, Plant K E Mol Cell Biol. 2005 April; 25(8):3276-85, Kynclová E Vnitr Lek. 1999 March; 45(3):151-4. Czech, Zhang Z Mol. Cell. 2004 Nov. 19; 16(4):597-607, Harteveld C L Hemoglobin. 2004 August; 28(3):255-9, Ling J J Biol. Chem. 2004 Dec. 3; 279(49):51704-13. Epub 2004 Oct. 1, Wachtel C RNA. 2004 November; 10(11):1740-50. Epub 2004 Sep. 23, Inácio A J Biol. Chem. 2004 Jul. 30; 279(31):32170-80. Epub 2004 May 25, Harteveld C L Am J Hematol. 2003 October; 74(2):99-103, Skabkina O V J Biol. Chem. 2003 May 16; 278(20):18191-8. Epub 2003 Mar. 19, Najmabadi H Haematologica. 2002 October; 87(10):1113-4. No abstract available, Viprakasit V Hemoglobin. 2002 May; 26(2):155-62, Sgourou A Br J. Haematol. 2002 August; 118(2):671-6, Moura G Yeast. 2002 Jun. 30; 19(9):727-33, Villemure J F J Mol. Biol. 2001 Oct. 5; 312(5):963-74, Bozdayi A M J Clin Virol. 2001 April; 21(1):91-101, Harteveld C L Haematologica. 2001 January; 86(1):36-8, Romão L Blood. 2000 Oct. 15; 96(8):2895-901, Gorman L J Biol. Chem. 2000 Nov. 17; 275(46):35914-9, Wang Z EMBO J. 2000 Jan. 17; 19(2):295-305, Razin S V J Cell Biochem. 1999 Jul. 1; 74(1):38-49, Dye M J Mol. Cell. 1999 March; 3(3):371-8, Chittum H S Biochemistry. 1998 Aug. 4; 37(31):10866-70, Thermann R EMBO J. 1998 Jun. 15; 17(12):3484-94, Norman J A Vaccine. 1997 June; 15(8):801-3, Oshima K Am J Hematol. 1996 May; 52(1):39-41, Yasunaga M Intern Med. 1995 December; 34(12):1198-200, Carter M S J Biol. Chem. 1995 Dec. 1; 270(48):28995-9003, Kobayashi M Mol Cell Probes. 1995 June; 9(3):175-82, Ellison J Biotechniques. 1994 October; 17(4):742-3, 746-7, 748-53, Angeloni S V Gene. 1994 Aug. 19; 146(1):133-4, Schiill C Nucleic Acids Res. 1994 Jun. 11; 22(11):1974-80, Divoky V Hum Genet. 1994 January; 93(1):77-8, Tantravahi J Mol Cell Biol. 1993 January; 13(1):578-87, Bailey A D J Biol. Chem. 1992 Sep. 15; 267(26):18398-406, Winichagoon P Biochim Biophys Acta. 1992 Aug. 25; 1139(4):280-6, Roberts S Genes Dev. 1992 August; 6(8):1562-74, Izban M G Genes Dev. 1992 July; 6(7):1342-56, Lim S K Mol Cell Biol. 1992 March; 12(3):1149-61, Safaya S Am J Hematol. 1992 March, 39(3):188-93, Riley J H Toxicol Pathol. 1992; 20(3 Pt 1):367-75, Ashfield R EMBO J. 1991 December; 10(13):4197-207, Enriquez-Harris P EMBO J. 1991 July; 10(7):1833-42, Wiest D K Mol Cell Biol. 1990 November; 10(11):5782-95, Müller H P Somat Cell Mol. Genet. 1990 July; 16(4):351-60, Briggs D Nucleic Acids Res. 1989 Oct. 25; 17(20):8061-71, Lim S EMBO J. 1989 September; 8(9):2613-9, Losekoot M Hum Genet. 1989 August; 83(1):75-8, Fucharoen S J Biol. Chem. 1989 May 15; 264(14):7780-3, Atweh G F J Clin Invest. 1988 August; 82(2):557-61, Logan J Proc Natl Acad Sci USA. 1987 December; 84(23):8306-10, Nakamura T Blood. 1987 September; 70(3):809-13, Shehee W R J Mol. Biol. 1987 Aug. 20; 196(4):757-67, Reines D J Mol. Biol. 1987 Jul. 20; 196(2):299-312, Stolle C A Blood. 1987 July; 70(1):293-300, Hess J J Mol Biol. 1985 Jul. 5; 184(1):7-21, Falck-Pedersen E Cell. 1985 April; 40(4):897-905, Weintraub H. Cell. 1983 April; 32(4):1191-203, Kinniburgh A J Nucleic Acids Res. 1982 Sep. 25; 10(18):5421-7, Tuite M F Mol Cell Biol. 1982 May; 2(5):490-7, Hansen J N J Biol. Chem. 1982 Jan. 25; 257(2):1048-52, Tuite M F J Biol. Chem. 1981 Jul. 25; 256(14):7298-304, Bienz M Nucleic Acids Res. 1980 Nov. 25; 8(22):5169-78, Chang J C Nature. 1979 Oct. 18; 281(5732):602-3, Shaw R F J Mol. Evol. 1977 May 13; 9(3):225-30 and Gesteland R F Cell. 1976 March; 7(3):381-90).

AcMNPV wild type and recombinant viruses replicate in a variety of insect cells, including continuous cell lines derived from the fall armyworm, Spodoptera frugiperda (Lepidoptera; Noctuidae). S. frugiperda (Sf) cells have a population doubling time of 18 to 24 hours and can be propagated in monolayer or in free suspension cultures. The preferred host cell line for protein production from recombinant baculoviruses is expresSF+ (SF+)®. SF+ are non-transformed, non-tumorigenic continuous cell lines derived from the fall armyworm, Spodoptera frugiperda (Lepidoptera; Noctuidae). SF+ are propagated at 28±2° C. without carbon dioxide supplementation. The preferred culture medium for SF+ cells is PSFM, a simple mixture of salts, vitamins, sugars and amino acids. No fetal bovine serum is used in cell propagation.

SF+ cells have a population doubling time of 18-24 hours and are propagated in free suspension cultures. S. frugiperda cells have not been reported to support the replication of any known mammalian viruses.

In other embodiments, host cells may be insect cell lines, such as caterpillar cells (see, e.g., Fung J C et al. J Ethnopharmacol. 2011 Oct. 31; 138(1):201-11. Epub 2011 Sep. 12, Lapointe J F et al. J Invertebr Pathol. 2011 November; 108(3):180-93. Epub 2011 Aug. 30, Micheloud G A et al. J Virol Methods. 2011 December; 178(1-2):106-16. Epub 2011 Aug. 30, Nguyen Q et al. J Virol Methods. 2011 August; 175(2):197-205. Epub 2011 May 17, Luo K et al. J Insect Sci. 2011; 11:6, Marchbank T et al. Br J Nutr. 2011 May; 105(9):1303-10. Epub 2011 Jan. 28, Tettamanti G et al. Methods Enzymol. 2008; 451:685-709, Kim H G et al. Eur J. Pharmacol. 2006 Sep. 18; 545(2-3):192-9. Epub 2006 Jun. 28, Lynn D E In Vitro Cell Dev Biol Anim. 2006 May-June; 42(5-6): 149-52, Mao W et al. Insect Mol. Biol. 2006 April; 15(2): 169-79. Erlandson M A et al. Can J Microbiol. 2006 March; 52(3):266-71, Waterfield N et al. Cell Microbiol. 2005 March, 7(3):373-82, McLean H et al. Insect Biochem Mol. Biol. 2005 January; 35(1):61-72, Wen Z et al. Insect Biochem Mol. Biol. 2003 September; 33(9):937-47, Miyata S et al. Infect Immun. 2003 May; 71(5):2404-13, Goodman C L et al. In Vitro Cell Dev Biol Anim. 2001 June; 37(6):374-9, Goodman C L et al. In Vitro Cell Dev Biol Anim. 2001 June; 37(6):367-73, Yazaki K et al. J Electron Microsc (Tokyo). 2000; 49(5):663-8, Maruniak J E et al. Arch Virol. 1999; 144(10):1991-2006, Wittwer D et al. Cytokine. 1999 September; 11(9):637-42, Hung C F et al. Insect Biochem Mol. Biol. 1997 May; 27(5):377-85. Castro M E et al. J Invertebr Pathol. 1997 January; 69(1):40-5, Bozon V et al. J Mol. Endocrinol. 1995 June; 14(3):277-84, Jahagirdar et al. Biochem Int. 1991 April; 23(6):1049-54, Klaiber K et al. Neuron. 1990 August; 5(2):221-6 And Ennis T J et al. Can J Genet Cytol. 1976 September; 18(3):471-7). The invention would particularly be applicable in insect cells susceptible to infection by AcMNPV.

In a particularly advantageous embodiment, the vectors of the present invention express an influenza exogenous gene. The influenza gene may express hemagglutinin, advantageously recombinant hemagglutinin, in particular, any recombinant influenza hemagglutinin (rHA). In particular, the rHA may be obtained from a strain formulated into a current influenza vaccine, such as H1 A/California/07/2009, H3 A/Victoria/361/2011, A/Texas/50/2012, B/Massachusetts/2/2012, A/Victoria/361/2011 and B: B/Wisconsin/1/2010-like; B/Hubei=alternative or Hubei-like (=B/Yamagata lineage), or A/Cal/ (influenza H1/California hemagglutinin). The rHA may also be part of a monovalent, divalent, trivalent or quadrivalent vaccine, which may include two B-strains, or a representative from each lineage: B/Victoria and B/Yamagata. In another embodiment, the rHA may be part of a monovalent, divalent, trivalent or quadrivalent, which may include combinations of other strains, such as, but not limited to, H1, H2, H3, H5, H7 and/or H9 strains.

Recombinant hemagglutinin antigens are expressed at high levels in S. frugiperda cells infected with AcNPV-hemagglutinin vectors. The primary gene product is unprocessed, full length hemagglutinin (rHA0) and is not secreted but remains associated with peripheral membranes of infected cells. This recombinant HA0 is a 68,000 molecular weight protein which is glycosylated with N-linked, high-mannose type glycans. There is evidence that rHA0 forms trimers post-translationally which accumulate in cytoplasmic membranes.

Post infection, rHA0 may be selectively extracted from the peripheral membranes of AcNPV-hemagglutinin infected cells with, for example, a non-denaturing, nonionic detergent or other methods known to those skilled in the art for purification of recombinant proteins from insect cells, including, but not limited to filtration and/or chromatography, such as affinity or other chromatography, and antibody binding. The detergent soluble rHA0 may be further purified, for example, using ion exchange and lectin affinity chromatography, or other equivalent methods known to those skilled in the art.

Purified rHA0 is resuspended in an isotonic, buffered solution. Following the removal of the detergent, purified rHA0 should efficiently agglutinate red blood cells if the rHA is functional.

rHA0 may be purified to at least 95% purity. This migrates predominantly as a single major polypeptide of 68,000 molecular weight on an SDS-polyacrylamide gel. The quaternary structure of purified recombinant HA0 antigen was examined by electron microscopy, trypsin resistance, density sedimentation analysis, and ability to agglutinate red blood cells. These data show that recombinant HA0 forms trimers and may assemble into rosettes.

The quantitative ability of purified rHA0 to agglutinate cells may be used as a measure of lot-to-lot consistency of the antigen. One hemagglutinin unit is defined as the quantity of antigen required to achieve 50% agglutination in a standard hemagglutinin assay with red blood cells, such as, but not limited to, chicken, guinea pig or hamster red blood cells. Comparative data shows that purified rHA0 antigens agglutinate red blood cells with an efficiency comparable to that observed with whole influenza virions.

The present invention may also express recombinant influenza hemagglutinin (rHA) from several influenza strains, including an H1 protein isolated from a California or Solomon strain (such as, but not limited to, a California/07/2009 strain or a Solomon Is/03/2006 strain), a B protein isolated from a Brisbane, Florida, Ohio, Jiangsu or Hong Kong strain (such as, but not limited to, a Brisbane/60/2008 strain, a Florida/04/2006 strain, an Ohio/01/2005 strain, a Jiangsu/10/2003 strain or a Hong Kong/330/2001 strain) or an H3 protein isolated from a Victoria, Perth, Bristane or Wisconsin strain (such as, but not limited to, a Victoria/361/2011 strain, a Perth/16/2009 strain, a Brisbane/16/2007 strain or a A/Wisconsin/67/05 strain). The present invention also contemplates mutant rHA from future influenza strains comprising cysteine mutations as disclosed herein.

Advantageously, the above-referenced proteins comprise one or more mutations. In particular, the one or more mutations are cysteine residues mutated to another residue. In an especially advantageous embodiment, the mutations may comprise mutations of one or more of the cysteine residues highlighted in FIG. 2.

Methods of generating mutations are well known to one of skill in the art. In a particular advantageous, but not limiting, embodiment, primers to generate C539A, C546A, C549A, C524A and C528A mutations in a H3 Perth rHA protein may comprise CCTTGCCATATCAgcTTTITTGCTTgcTGTTGCTITGTTGGGG as a forward primer and CCCCAACAAAGCAACAgcAAGCAAAAAAgcTGATATGGCAAAGG as a reverse primer. In another advantageous embodiment, primers to generate C539A, C546A and C549A mutations in a H3 Perth rHA protein may comprise GGGGTTCATCATGTGGGCCgcCCAAAAAGGCAACATTAGGgcCAACATTgcCATTTAA GTAAGTACCG as a forward primer and CGGTACTTACTTAAATGgcAATGTTGgcCCTAATGTTGCClTTTGGgcGGCCCACATG ATGAACCCC as a reverse primer. In another advantageous embodiment, primers to generate C524S and C528A mutations in a H3 Perth rHA protein may comprise CCTTGCCATATCATcTlTITTGCTTgcTGTTGCTTTGTTGGGG as a forward primer and CCCCAACAAAGCAACAgcAAGCAAAAAAgATGATATGGCAAAGG as a reverse primer.

In another embodiment, the influenza exogeneous gene may include any other influenza protein.

Examples of other influenza strains include, but are not limited to, turkey influenza virus strain A/Turkey/Ireland/1378/83 (H5N8) (see, e.g., Taylor et al., 1988b), turkey influenza virus strain A/Turkey/England/63 (H7N3) (see, e.g., Alexander et al., 1979; Rott et al., 1979; Horimoto et al., 2001), turkey influenza virus strain A/Turkey/England/66 (H6N2) (see, e.g., Alexander et al., 1979), A/Turkey/England/69 (H7N2) (see, e.g., Alexander et al., 1979; Horimoto et al., 2001), A/Turkey/Scotland/70 (H6N2) (see, e.g., Banks et al., 2000; Alexander et al., 1979), turkey influenza virus strain A/Turkey/England N28/73 (H5N2) (see, e.g., Alexander et al., 1979), turkey influenza virus strain A/Turkey/England/110/77 (H6N2) (see, e.g., Alexander et al., 1979), turkey influenza virus strain A/Turkey/England/647/77 (H1N1) (see, e.g., Alexander et al., 1979; Karasin et al., 2002)), turkey influenza virus strain A/Turkey/Ontario/7732/66 (H5N9) (see, e.g., Slemons et al., 1972; Philpott et al., 1989), turkey influenza virus strain A/Turkey/England/199/79 (H7N7) (see, e.g., Horimoto et al., 2001), turkey influenza virus strain A/Turkey/Ontario/7732/66 (H5N9) (see, e.g., Horimoto et al., 2001; Panigrahy et al., 1996), turkey influenza virus strain A/Turkey/Ireland/1378/85 (H5N8) (see, e.g., Horimoto et al., 2001; Walker et al., 1993), turkey influenza virus strain A/Turkey/England/50-92/91 (H5N1) (see, e.g., Horimoto et al., 2001; Howard et al., 2006), turkey influenza virus strain A/Turkey/Wisconsin/68 (H5N9), turkey influenza virus strain A/Turkey/Massachusetts/65 (H6N2), turkey influenza virus strain A/Turkey/Oregon/71 (H7N3), (see, e.g., Orlich et al., 1990), turkey influenza virus strain A/Turkey/Ontario/6228/67 (H8N4), turkey influenza virus strain A/Turkey/Wisconsin/66 (H9N2), (see, e.g., Zakstel'skaia et al., 1977), turkey influenza virus strain A/Turkey/England/647/77 (H1N1) (see, e.g., Karasin et al., 2002; Alexander et al., 1979), turkey influenza virus strain A/Turkey/Ontario/6118/68 (H8N4) (see, e.g., Blok et al., 1982), turkey influenza virus strain A/Tur/Ger 3/91 (see, e.g., Zakay-Rones et al., 1995), turkey influenza virus strain A/Turkey/Minnesota/833/80 (H4N2) (see, e.g., Gubareva et al., 1997) chicken influenza virus strain A/Chicken/Indonesia/03 (H5N1), chicken influenza virus strain A/Chicken/FPV/Rostock/1934 (see, e.g., Ohuchi et al., 1994), chicken influenza virus strain A/Chicken/Texas/298313/04 (see, e.g., Lee et al., 2005), chicken influenza virus strain A/Chicken/Texas/167280-4-/02 (see, e.g., Lee et al., 2005), chicken influenza virus strain A/Chicken/Hong Kong/220/97 (see, e.g., Perkins et al., 2001), chicken influenza virus strain A/Chicken/Italy/8/98 (see, e.g., Capua et al., 1999), chicken influenza virus strain A/Chicken/Victoria/76 (H7N7) (see, e.g., Zambon, 2001; Nestorowicz et al., 1987), chicken influenza virus strain A/Chicken/Germany/79 (H7N7) (see, e.g., Rohm et al., 1996), chicken influenza virus strain A/Chicken/Scotland/59 (H5N1) (see, e.g., Horimoto et al., 2001; De et al., 1988; Wood et al., 1993), chicken influenza virus strain A/Chicken/Pennsylvania/1370/83 (H5N2) (see, e.g., Bean et al., 1985; van der Goot et al., 2002), chicken influenza virus strain A/Chicken/Queretaro-19/95 (H5N2) (see, e.g., Horimoto et al., 2001; Garcia et al., 1998), chicken influenza virus strain A/Chicken/Queretaro-20/95 (H5N2) (see, e.g., Horimoto et al., 2001), chicken influenza virus strain A/Chicken/Hong Kong/258/97 (H5N1) (see, e.g., Horimoto et al., 2001; Webster, 1998), chicken influenza virus strain A/Chicken/Italy/1487/97 (H5N2) (see, e.g., Horimoto et al., 2001), chicken influenza virus strain A/Chicken/Leipzig/79 (H7N7) (see, e.g., Horimoto et al., 2001; Rohm et al., 1996), chicken influenza virus strain A/Chicken/Victoria/185 (H7N7) (see, e.g., Horimoto et al., 2001), chicken influenza virus strain A/Chicken/Victoria/92 (H7N3) (see, e.g., Horimoto et al., 2001), chicken influenza virus strain A/Chicken/Queensland/95 (H7N3) (see, e.g., Horimoto et al., 2001), chicken influenza virus strain A/Chicken/Pakistan/1369/95 (H7N2) (see, e.g., Horimoto et al., 2001), chicken influenza virus strain A/Chicken/Pakistan/447-4/95 (H7N3) (see, e.g., Horimoto et al., 2001), chicken influenza virus strain A/Chicken/HK/G9/97 (H9N2) (see, e.g., Leneva et al., 2001), chicken influenza virus strain A/Chicken/Nakom-Patom/Thailand/CU-K2/2004(H5N1) (see, e.g., Anwar et al., 2006; Viseshakul et al., 2004), chicken influenza virus strain A/Chicken/Hong Kong/31.2/2002 (H5N11), (see, e.g., Anwar et al., 2006), chicken influenza virus strain A/Chicken/Vietnam/C58/04 (H5N1), (see, e.g., Anwar et al., 2006), chicken influenza virus strain A/Chicken/Vietnam/38/2004(H5N1), (see, e.g., Anwar et al., 2006), chicken influenza virus strain A/Chicken/Alabama/7395/75 (H4N8), (see, e.g., Swayne et al., 1994), chicken influenza virus strain A/Chicken/Germany/N/49 (H100N7), (see, e.g., Yamane et al., 1981), chicken influenza virus strain A/Chicken/Beijing/1/94 (H9N2) (see, e.g., Karasin et al., 2002), chicken influenza virus strain A/Chicken/Hong Kong/G23/97 (H9N2) (see, e.g., Karasin et al., 2002), chicken influenza virus strain A/Chicken/Pennsylvania/8125/83 (H5N2) (see, e.g., Karasin et al., 2002; Shortridge et al., 1998), chicken influenza virus strain A/Chicken/Hong Kong/97 (H5N1) (see, e.g., Chen et al., 2003), duck influenza virus strain A/Duck/Anyang/AVL-1/01 (see, e.g., Tumpey et al., 2002), duck influenza virus strain A/Duck/New York/17542-4/86 (H9N1) (see, e.g., Banks et al., 2000), duck influenza virus strain A/Duck/Alberta/28/76 (H4N6) (see, e.g., Blok et al., 1982), duck influenza virus strain A/Duck/Nanchang/4-165/2000 (H4N6) (see, e.g., Liu et al., 2003), duck influenza virus strain A/Duck/Germany/49 (H10N7) (see, e.g., Blok et al., 1982), duck influenza virus strain A/Black Duck/Australia/702/78 (H3N8) (see, e.g., Blok et al., 1982), duck influenza virus strain A/Duck/Vietnam/11/2004 (H5N1), (see, e.g., Anwar et al., 2006), duck influenza virus strain A/Duck/Alberta/60/76 (H12N5), (see, e.g., Baez et al., 1981), duck influenza virus strain A/Duck/Hong Kong/196/77 (H1) (see, e.g., Karasin et al., 2002; Kanegae et al., 1994), duck influenza virus strain A/Duck/Wisconsin/1938/80 (H1N1) (see, e.g., Karasin et al., 2002), duck influenza virus strain A/Duck/Bavaria/2/77 (H1N1N1) (see, e.g., Karasin et al., 2002; Ottis et al., 1980), duck influenza virus strain A/Duck/Bavaria/1/77 (H1N1) (see, e.g., Ottis et al., 1980), duck influenza virus strain A/Duck/Australia/749/80 (H1N1) (see, e.g., Karasin et al., 2002), duck influenza virus strain A/Duck/Hong Kong/Y280/97 (H9N2) (see, e.g., Karasin et al., 2002; Guan et al., 2000), duck influenza virus strain A/Duck/Alberta/35/76 H1N1) (see, e.g., Austin et al., 1990), avian influenza virus strain A/Mallard duck/Gurjev/263/82 (H14N5), (see, e.g., Kawaoka et al., 1990), avian influenza virus strain A/Mallard duck/PA/10218/84 (H5N2) (see, e.g., Smirnov et al., 2000), avian influenza virus strain A/Mallard duck/Astrakhan/244/82 (H14N6) (see, e.g., Karasin et al., 2002), goose influenza virus strain A/Goose/Guangdong/1/96 (see, e.g., Xu et al., 1999), goose influenza virus strain A/Goose/Leipzig/137-8/79 (H7N7) (see, e.g., Horimoto et al., 2001), goose influenza virus strain A/Goose/Hong Kong/W222/97 (H6N7) (see, e.g., Chin et al., 2002), goose influenza virus strain A/Goose/Leipzig/187-7/79 (H7N7) (see, e.g., Horimoto et al., 2001), goose influenza virus strain A/Goose/Leipzig/192-7/79 (H7N7) (see, e.g., Horimoto et al., 2001), avian influenza virus strain A/Env/HK/437-4/99 (see, e.g., Cauthen et al., 2000), avian influenza virus strain A/Env/HK/437-6/99 (see, e.g., Cauthen et al., 2000), avian influenza virus strain A/Env/HK/437-8/99 (see, e.g., Cauthen et al., 2000), avian influenza virus strain A/Env/HK/437-10/99, (see, e.g., Cauthen et al., 2000), avian influenza virus strain A/Fowl plague virus strain/Dutch/27 (H7N7) (see, e.g., Horimoto et al., 2001; Carter et al., 1982), avian influenza virus strain A/Fowl plague virus strain/Dobson/27 (H7N7) (see, e.g., Horimoto et al., 2001), avian influenza virus strain A/Fowl plague virus strain/Rostock/34 (H7N1) (see, e.g., Horimoto et al., 2001; Takeuchi et al., 1994), avian influenza virus strain A/Fowl plague virus strain/Egypt/45 (H7N1) (see, e.g., Horimoto et al., 2001), avian influenza virus strain A/Fowl plague virus strain/Weybridge (H7N7) (see, e.g., Tonew et al., 1982), avian influenza virus strain A/Tem/South Africa/61 (H5N3) (see, e.g., Horimoto et al., 2001; Perkins et al., 2002; Walker et al., 1992), avian influenza virus strain A/Tern/Australia/G70C/75 (H11N9) (see, e.g., Pruett et al., 1998), avian influenza virus strain A/Quail/Vietnam/36/04(H5N1), (see, e.g., Anwar et al., 2006), avian influenza virus strain A/Gull/Maryland/704/77 (H13N6), (see, e.g., Iamnikova et al., 1989), avian influenza virus strain A/Black-headed gull/Sweden/5/99 (H16N3) (see, e.g., Fouchier et al., 2005), avian influenza virus strain A/Herring gull/DE/677/88 (H2N8) (see, e.g., Saito et al., 1993), avian influenza virus strain A/Swan/Italy/179/06 (H5N1) (see, e.g., Terregino et al., 2006), avian influenza virus strain A/Hong Kong/156/97 (A/HK/156/97) (see, e.g., Leneva et al., 2001; Claas et al., 1998; Cauthen et al., 2000), avian influenza virus strain A/Quail/HK/G1/97 (H9N2) (see, e.g., Leneva et al., 2001), avian influenza virus strain A/Quail/Hong Kong/AF157/93 (H9N2) (see, e.g., Karasin et al., 2002), avian influenza virus strain A/Teal/HK/W312/97 (H6N1) (see, e.g., Leneva et al., 2001), avian influenza virus strain A/Shearwater/West Australia/2576/79 (H15N9) (see, e.g., Rohm et al., 1996), avian influenza virus strain A/Shearwater/Australia/72 (H6N5) (see, e.g., Harley et al., 1990), avian influenza virus strain A/Hong Kong/212/03 (see, e.g., Shinya et al., 2005), avian influenza virus strain A/England/321/77 (H3N2) (see, e.g., Hauptmann et al., 1983), avian pandemic influenza A viruses of avian origin (see, e.g., Audsley et al., 2004) avian H5N1 influenza virus, avian H7N1 influenza strain (see, e.g., Foni et al., 2005), avian H9N2 influenza virus (see, e.g., Leneva et al., 2001), and avian influenza virus, cold-adapted (ca) and temperature sensitive (ts) master donor strain, A/Leningrad/134/17/57 (H2N2) (see, e.g., Youil et al., 2004), the disclosures of which are incorporated by reference.

Other influenza strains that may be used in methods of the present invention include, but are not limited to, equine influenza virus (A/Equi 2 (H3N8), Newmarket 1/93) (see, e.g., Mohler et al., 2005; Nayak et al., 2005), equine-2 influenza virus (EIV; subtype H3N8) (see, e.g., Lin et al., 2001), equine-2 influenza virus, A/Equine/Kentucky/1/91 (H3N8) (see, e.g., Youngner et al., 2001), equine influenza virus strain A/Equine/Berlin/2/91 (H3N8) (see, e.g., Ilobi et al., 1998), equine influenza virus strain A/Equine/Cambridge/1/63 (H7N7) (see, e.g., Gibson et al., 1992), equine influenza virus strain A/Equine/Prague/1/56 (H7N7) (see, e.g., Karasin et al., 2002; Appleton et al., 1989), equine influenza virus strain A/Eq/Kentucky/98 (see, e.g., Crouch et al., 2004), equine influenza virus strain A/Equi 2 (Kentucky 81) (see, e.g., Short et al., 1986; Horner et al., 1988), equine influenza virus strain A/Equine/Kentucky/1/81 (Eq/Ky) (see, e.g., Breathnach et al., 2004), equine influenza virus strain A/Equine Kentucky/1/81 (H3N8) (see, e.g., Olsen et al., 1997; Morley et al., 1995; Ozaki et al., 2001; Sugiura et al., 2001; Goto et al., 1993), equine influenza virus strain A/Equine/Kentucky/1/91 (H3N8) (see, e.g., Youngner et al., 2001), equine influenza virus strain A/Equine/Kentucky/1277/90 (Eq/Kentucky) (see, e.g., Webster et al., 1993), equine influenza virus strain A/Equine/Kentucky/2/91 (H3N8) (see, e.g., Donofrio et al., 1994), equine influenza virus strain A/Equine/Kentucky/79 (H3N8) (see, e.g., Donofrio et al., 1994), equine influenza virus strain A/Equine/Kentucky/81 (see, e.g., Sugiura et al., 2001), equine influenza virus strain A/Equine/Kentucky/91 (H3N8) (see, e.g., Gross et al., 1998), equine influenza virus strain A/Equine-2/Kentucky/95 (H3N8) (see, e.g., Heldens et al., 2004) and equine influenza virus strain A/Equine-2/Kentucky/98 (see, e.g., Chambers et al., 2001), equine influenza virus strain A/Eq/Newmarket/1/77 (see, e.g., Lindstrom et al., 1998), equine influenza virus strain A/Eq/Newmarket/5/03 (see, e.g., Edlund Toulemonde et al., 2005), equine influenza virus strain A/Equi 2 (H3N8), Newmarket 1/93 (see, e.g., Mohler et al., 2005; Nayak et al., 2005), equine influenza virus strain A/Equi-2/Newmarket-1/93 (see, e.g., Heldens et al., 2002), equine influenza virus strain A/Equine/Newmarket/2/93 (see, e.g., Wattrang et al., 2003), equine influenza virus strain A/Equine/Newmarket/79 (H3N8) (see, e.g., Duhaut et al., 2000; Noble et al., 1994; Duhaut et al., 1998; Hannant et al., 1989; Hannant et al., 1989; Hannant et al., 1988; Richards et al., 1992; Heldens et al., 2004), equine influenza virus strain A/Equine/Newmarket/1/77 (H7N7) (see, e.g., Goto et al., 1993; Sugiura et al., 2001) and equine influenza virus strain A/Equine-2/Newmarket-2/93 (see, e.g., Heldens et al., 2004), equine influenza virus strain A/Eq/Miami/63 (H3N8) (see, e.g., van Maanen et al., 2003), A/Equi 1 (Prague strain) (see, e.g., Horner et al., 1988; Short et al., 1986), equine influenza virus strain A/Equi 2 (Miami) (see, e.g., Short et al., 1986), equine influenza virus strain A/Equi-1/Prague/56 (Pr/56) (see, e.g., Heldens et al., 2002), equine influenza virus strain A/Equi-2/Suffolk/89 (Suf/89) (see, e.g., Heldens et al., 2002), equine influenza virus strain A/Equine 2/Sussex/89 (H3N8) (see, e.g., Mumford et al., 1994), equine influenza virus strain A/Equine/Sussex/89 (see, e.g., Wattrang et al., 2003), equine influenza virus strain A/Equine-2/Saskatoon/90 (see, e.g., Chambers et al., 2001), equine influenza virus strain A/Equine/Prague/1/56 (H7N7) (see, e.g., Donofrio et al., 1994; Morley et al., 1995), equine influenza virus strain A/Equine/Miami/1/63 (H3N8) (see, e.g., Morley et al., 1995; Ozaki et al., 2001; Thomson et al., 1977; Mumford et al., 1988; Donofrio et al., 1994; Mumford et al., 1983), A/Aichi/2/68 (H3N2) (see, e.g., Ozaki et al., 2001), equine influenza virus strain A/Equine/Tokyo/2/71 (H3N8) (see, e.g., Goto et al., 1993), equine influenza virus strain A/Eq/LaPlata/1/88 (see, e.g., Lindstrom et al., 1998), equine influenza virus strain A/Equine/Jilin/1/89 (Eq/Jilin) (see, e.g., Webster et al., 1993), equine influenza virus strain A/Equine/Alaska/1/91 (H3N8) (see, e.g., Webster et al., 1993), equine influenza virus strain A/Equine/Saskatoon/1/91 (H3N8) (see, e.g., Morley et al., 1995), equine influenza virus strain A/Equine/Rome/5/91 (H3N8) (see, e.g., Sugiura et al., 2001), equine influenza virus strain A/Equine/La Plata/1/93 (H3N8) (see, e.g., Ozaki et al., 2001), equine influenza virus strain A/Equine/La Plata/1/93 (LP/93) (see, e.g., Sugiura et al., 2001), equine influenza virus strain A/Eq/Holland/1/95 (H3N8) (see, e.g., van Maanen et al., 2003) and equine influenza virus strain A/Eq/Holland/2/95 (H3N8) (see, e.g., van Maanen et al., 2003), human influenza virus A(H3N2) isolates (see, e.g., Abed et al., 2002), human influenza virus A/Memphis/1/71 (H3N2) (see, e.g., Suzuki et al., 1996), human influenza virus A/Nanchang/933/95 (H3N2) virus (see, e.g., Scholtissek et al., 2002), human influenza virus A/PR/8/34 (H1N1) virus (see, e.g., Scholtissek et al., 2002), human influenza virus A/Singapore/57 (H2N2) virus (see, e.g., Scholtissek et al., 2002), influenza virus A (see, e.g., Chare et al., 2003), influenza virus A/HK/213/03 (see, e.g., Guan et al., 2004; Anwar et al., 2006), influenza virus strain A/HK/483/97 (see, e.g., Cheung et al., 2002), influenza virus strain A/HK486/97 (see, e.g., Cheung et al., 2002), influenza virus strain A/Thailand/5(KK-494)/2004 (H5N1), (see, e.g., Anwar et al., 2006), influenza virus strain A PR/8/34 (PR8) virus strain (H1N1 subtype) (see, e.g., Mantani et al., 2001), influenza virus strain A/Aichi/2/68(H3N2) (see, e.g., Miyamoto et al., 1998), influenza virus strain A/Ann Arbor/6/60 cold-adapted virus strain (see, e.g., Treanor et al., 1994), influenza virus strain A/Beijing 32/92 (H3N2) (see, e.g., Zakay-Rones et al., 1995), influenza virus strain A/Charlottesville/31/95 (H1N1) (see, e.g., Gubareva et al., 2002), influenza virus strain A/Kawasaki/86 (H1N1) virus strain (see, e.g., Staschke et al., 1998), influenza virus strain A/Korea/82 (H3N2) (see, e.g., Treanor et al., 1994), influenza virus strain A/Leningrad/134/57 (see, e.g., Egorov et al., 1998), influenza virus strain A/NWS/33 (H1N1) (see, e.g., Sidwell et al., 1998), influenza virus strain A/PR/8/34(H1N1) (see, e.g., Miyamoto et al., 1998), influenza virus strain A/PR8/34 (see, e.g., Nunes-Correia et al., 1999; Tree et al., 2001), influenza virus strain A/Puerto Rico (PR)/8/34 (see, e.g., Egorov et al., 1998), influenza virus strain A/Puerto Rico/8-Mount Sinai (see, e.g., Mazanec et al., 1995), influenza virus strain A/Shangdong 9/93 (H3N2) (see, e.g., Zakay-Rones et al., 1995; Sidwell et al., 1998), influenza virus strain A/Shingapol/1/57(H2N2) (see, e.g., Miyamoto et al., 1998), influenza virus strain A/Singapore 6/86 (H1N1) (see, e.g., Zakay-Rones et al., 1995), influenza virus strain A/Singapore/1/57 (H2N2) (see, e.g., Bantia et al., 1998), influenza virus strain A/Texas 36/91 (H1N1) (see, e.g., Zakay-Rones et al., 1995), influenza virus strain A/Texas/36/91 (H1N1) virus strain (see, e.g., Gubareva et al., 2001; Halperin et al., 1998), influenza virus strain A/Texas/36/91(H1N1) (see, e.g., Hayden et al., 1994), influenza virus strain A/Udorn/72 virus infection (see, e.g., Shimizu et al., 1999), influenza virus A/Victoria/3/75 (H3N2) (see, e.g., Sidwell et al., 1998), influenza virus A/Virginia/88(H3N2) (see, e.g., Hayden et al., 1994), influenza virus A/WSN/33 (H1N1) (see, e.g., Lu et al., 2002), influenza virus A/WSN/33 (see, e.g., Gujuluva et al., 1994), influenza virus B (see, e.g., Chare et al., 2003), influenza virus B/Ann Arbor 1/86 (see, e.g., Zakay-Rones et al., 1995), influenza virus B/Harbin/7/94 (see, e.g., Halperin et al., 1998), influenza virus B/Hong Kong/5/72 (see, e.g., Sidwell et al., 1998), influenza virus B/Lee/40 (see, e.g., Miyamoto et al., 1998), influenza virus B/Victoria group (see, e.g., Nakagawa et al., 1999), influenza virus B/Yamagata 16/88 (see, e.g., Zakay-Rones et al., 1995), influenza virus B/Yamagata group (see, e.g., Nakagawa et al., 1999), influenza virus B/Yamanashi/166/98 (see, e.g., Hoffmann et al., 2002), influenza virus C (see, e.g., Chare et al., 2003), influenza virus strain A/Equi/2/Kildare/89 (see, e.g., Quinlivan et al., 2004), influenza virus type B/Panama 45/90 (see, e.g., Zakay-Rones et al., 1995), live, cold-adapted, temperature-sensitive (ca/ts) Russian influenza A vaccines (see, e.g., Palker et al., 2004), swine H1 and H3 influenza viruses (see, e.g., Gambaryan et al., 2005), swine influenza A viruses (see, e.g., Landolt et al., 2005), swine influenza virus (SIV) (see, e.g., Clavijo et al., 2002), swine influenza virus A/Sw/Ger 2/81 (see, e.g., Zakay-Rones et al., 1995), swine influenza virus A/Sw/Ger 8533/91 (see, e.g., Zakay-Rones et al., 1995), swine influenza virus strain A/Swine/Wisconsin/125/97 (H1N1) (see, e.g., Karasin et al., 2002; Karasin et al., 2006), swine influenza virus strain A/Swine/Wisconsin/136/97 (H1N1) (see, e.g., Karasin et al., 2002), swine influenza virus strain A/Swine/Wisconsin/63/97 (H1N1) (see, e.g., Karasin et al., 2002), swine influenza virus strain A/Swine/Wisconsin/164/97 (H1N1) (see, e.g., Karasin et al., 2002), swine influenza virus strain A/Swine/Wisconsin/166/97 (H1N1) (see, e.g., Karasin et al., 2002), swine influenza virus strain A/Swine/Wisconsin/168/97 (H1N1) (see, e.g., Karasin et al., 2002), swine influenza virus strain A/Swine/Wisconsin/235/97 (H1N1) (see, e.g., Karasin et al., 2002; Olsen et al., 2000), swine influenza virus strain A/Swine/Wisconsin/238/97 (H1N1) (see, e.g., Karasin et al., 2002; Ayora-Talavera et al., 2005), swine influenza virus strain A/Swine/Wisconsin/457/98 (H1N1) (see, e.g., Karasin et al., 2002), swine influenza virus strain A/Swine/Wisconsin/458/98 (H1N1) (see, e.g., Karasin et al., 2002; Karasin et al., 2006), swine influenza virus strain A/Swine/Wisconsin/464/98 (H1N1) (see, e.g., Karasin et al., 2002; Karasin et al., 2006), swine influenza virus strain A/Swine/Indiana/1726/88 (H1N1) (see, e.g., Karasin et al., 2002; Macklin et al., 1998), swine influenza virus strain A/Swine/Indiana/9K035/99 (H N2) (see, e.g., Karasin et al., 2002; Karasin et al., 2000), swine influenza virus strain A/Swine/Nebraska/1/92 (H1N1) (see, e.g., Karasin et al., 2002), swine influenza virus strain A/Swine/Quebec/91 (H1N1) (see, e.g., Karasin et al., 2002), swine influenza virus strain A/Swine/Quebec/81 (H N1) (see, e.g., Karasin et al., 2002), swine influenza virus strain A/Swine/New Jersey/11/76 (H1N1) (see, e.g., Karasin et al., 2002), swine influenza virus strain A/Swine/Ehime/1/80 (H1N2) (see, e.g., Karasin et al., 2002; Nerome et al., 1985), swine influenza virus strain A/Swine/England/283902/93 (H1N1) (see, e.g., Karasin et al., 2002), swine influenza virus strain A/Swine/England/195852/92 (H1N1) (see, e.g., Karasin et al., 2002; Brown et al., 1993), swine influenza virus strain A/Swine/Germany/8533/91 (H1N1) (see, e.g., Karasin et al., 2002), swine influenza virus strain A/Swine/Germany/2/81 (H1N1) (see, e.g., Karasin et al., 2002), swine influenza virus strain A/Swine/Nebraska/209/98 (H3N2) (see, e.g., Karasin et al., 2002), A/Swine/Iowa/533/99 (H3N2) (see, e.g., Karasin et al., 2002), swine influenza virus strain A/Swine/Iowa/569/99 (H3N2) (see, e.g., Karasin et al., 2002), swine influenza virus strain A/Swine/Minnesota/593/99 (H3N2) (see, e.g., Karasin et al., 2002; Ayora-Talavera et al., 2005), swine influenza virus strain A/Swine/Iowa/8548-1/98 (H3N2) (see, e.g., Karasin et al., 2002), swine influenza virus strain A/Swine/Minnesota/9088-2/98 (H3N2) (see, e.g., Karasin et al., 2002), swine influenza virus strain A/Swine/Texas/4199-2/98 (H3N2) (see, e.g., Karasin et al., 2002), swine influenza virus strain A/Swine/Ontario/41848/97 (H3N2) (see, e.g., Karasin et al., 2002), swine influenza virus strain A/Swine/North Carolina/35922/98 (H3N2) (see, e.g., Karasin et al., 2002), A/Swine/Colorado/1/77 (H3N2) (see, e.g., Karasin et al., 2002), swine influenza virus strain A/Swine/Hong Kong/3/76 (H3N2) (see, e.g., Karasin et al., 2002), swine influenza virus strain A/Swine/Hong Kong/13/77 (H3N2) (see, e.g., Karasin et al., 2002), swine influenza virus strain A/Swine/Nagasaki/1/90 (H1N2) (see, e.g., Karasin et al., 2002), swine influenza virus strain A/Swine/Nagasaki/1/89 (H1N2) (see, e.g., Karasin et al., 2002), swine influenza virus strain A/Swine/Wisconsin/1915/88 (H1N1) (see, e.g., Karasin et al., 2002), swine influenza virus strain A/Swine/Iowa/17672/88 (H1N1) (see, e.g., Karasin et al., 2002), swine influenza virus strain A/Swine/Tennessee/24/77 (H1N1) (see, e.g., Karasin et al., 2002), swine influenza virus strain A/Swine/Ontario/2/81 (H N1) (see, e.g., Karasin et al., 2002), swine influenza virus strain A/Swine/Wisconsin/1/67 (H1N1) (see, e.g., Karasin et al., 2002), swine influenza virus strain A/Swine/Italy/1521/98 (H1N2) (see, e.g., Marozin et al., 2002), swine influenza virus strain A/Swine/Italy/839/89 (H1N1) (see, e.g., Karasin et al., 2002), swine influenza virus strain A/Swine/Hong Kong/126/82 (H3N2) (see, e.g., Karasin et al., 2002), influenza virus strain A/Idaho/4/95 (H3N2) (see, e.g., Karasin et al., 2002), influenza virus strain A/Johannesburg/33/94 (H3N2) (see, e.g., Karasin et al., 2002; Johansson et al., 1998), influenza virus strain A/Bangkok/1/79 (H3N2) (see, e.g., Karasin et al., 2002; Nelson et al., 2001), influenza virus strain A/Udorn/72 (H3N2) (see, e.g., Karasin et al., 2002; Markoff et al., 1982), influenza virus strain A/Hokkaido/2/92 (H1N1) (see, e.g., Karasin et al., 2002), influenza virus strain A/Thailand/KAN-1/04 (see, e.g., Puthavathana et al., 2005; Amonsin et al., 2006), influenza virus strain A/England/1/53 (see, e.g., Govorkova E A, et al., 1995), influenza virus strain A/Vietnam/3046/2004 (H5N1), (see, e.g., Anwar et al., 2006), influenza virus strain A/Vietnam/1203/2004 (H5N1), (see, e.g., Anwar et al., 2006; Gao et al., 2006), influenza virus strain A/tiger/Thailand/SPB-1(H5N1), (see, e.g., Anwar et al., 2006), influenza virus strain A/Japan/305/57 (H2N2) (see, e.g., Naeve et al., 1990; Brown et al., 1982), influenza virus strain A/Adachi/2/57 (H2N2) (see, e.g., Gething et al., 1980), influenza virus strain A/Camel/Mongolia/82 (H1N1) (see, e.g., Yamnikova et al., 1993), influenza virus strain A/RI/5/57 (H2N2) (see, e.g., Elleman et al., 1982), influenza virus strain A/Whale/Maine/1/84 (H13N9) (see, e.g., Air et al., 1987), influenza virus strain A/Taiwan/1/86 (H1N1) (see, e.g., Karasin et al., 2002; Brown, 1988), influenza virus strain A/Bayern/7/95 (H N1) (see, e.g., Karasin et al., 2002), influenza virus strain A/USSR/90/77 (H1N1) (see, e.g., Karasin et al., 2002; Iftimovici et al., 1980), influenza virus strain A/Wuhan/359/95 (H3N2) (see, e.g., Karasin et al., 2002; Hardy et al., 2001), influenza virus strain A/Hong Kong/5/83 (H3N2) (see, e.g., Karasin et al., 2002), influenza virus strain A/Memphis/8/88 (H3N2) (see, e.g., Karasin et al., 2002; Hatta et al., 2002), influenza virus strain A/Beijing/337/89 (H3N2) (see, e.g., Karasin et al., 2002), influenza virus strain A/Shanghai/6/90 (H3N2) (see, e.g., Karasin et al., 2002), influenza virus strain A/Akita/1/94 (H3N2) (see, e.g., Karasin et al., 2002), influenza virus strain A/Akita/11/95 (H3N2) (see, e.g., Karasin et al., 2002), influenza virus strain A/Memphis/6/90 (H3N2) (see, e.g., Karasin et al., 2002), influenza virus strain A/Udorn/307/72 (H3N2) (see, e.g., Karasin et al., 2002; Iuferov et al., 1984), influenza virus strain A/Singapore/1/57 (H2N2) (see, e.g., Karasin et al., 2002; Zhukova et al., 1975), influenza virus strain A/Ohio/4/83 (H1N1) (see, e.g., Karasin et al., 2002), influenza virus strain Madin Darby Canine Kidney (MDCK)-derived cell line (see, e.g., Halperin et al., 2002), mouse-adapted influenza virus strain A/Guizhou/54/89 (H3N2 subtype) (see, e.g., Nagai et al., 1995), mouse-adapted influenza virus A/PR/8/34 (A/PR8) (see, e.g., Nagai et al., 1995), mouse-adapted influenza virus B/Ibaraki/2/85 (see, e.g., Nagai et al., 1995). Russian live attenuated influenza vaccine donor strains A/Leningrad/134/17/57, A/Leningad/134/47/57 and B/USSR/60/69 (see, e.g., Audsley et al. 2005), the disclosures of which are incorporated by reference.

The present invention relates to methods for stabilizing protein vaccines which may comprise adding an antioxidant and a low toxicity reducing agent.

In one embodiment, the antioxidant may advantageously be citrate. Citrate can be in the form of a salt having one, two, or three positive counterions, or cations. Cations can be monatomic or polyatomic. Examples of suitable cations for citrate include, but are not limited, alkali metal cations, alkaline earth metal cations, transition metal cations and ammonium cations. Examples of suitable alkali metal cations include, but are not limited, Na⁺, K⁺, Li⁺, and the like. Examples of suitable alkaline earth metal cations include, but are not limited to, Ca²⁺, Mg²⁺, and the like. Examples of suitable transition metal cations include, but are not limited, Fe³⁺, Zn²⁺, and the like. The counterions in citrate can be the same or different. For example, a citrate may have ammonium (NH₄⁺) cations and ferric (Fe³⁺) cations, such as ammonium ferric citrate. A citrate may refer either to the conjugate base of citric acid, (C₃H₅O(COO)₃³⁻), or to the esters of citric acid. The citrate may be a salt, such as monosodium citrate, disodium citrate or trisodium citrate. The citrate may also be food additive E331. In another embodiment, the citrate may be an ester, such as triethyl citrate.

Generally, an antioxidant contemplated for the present invention may be any reducing agent such as a thiol, ascorbic acid, or a polyphenol or any derivative thereof. For example, antioxidant may be, but not limited to, ascorbate, tocopherols, carotenoids, butylhydroxytoluene (BHT), butylated hydroxyanisole (BHA) or lactate.

Thioglycolate is the conjugate base of thioglycolic acid, HSCH₂CO₂H. Thioglycolate can be in the form of a salt having at least one positive counterion, or cations. Cations can be monatomic or polyatomic. Examples of suitable cations for thioglycolate include, but are not limited, alkali metal cations, alkaline earth metal cations, transition metal cations and ammonium (NH₄⁺) cations. Examples of suitable alkali metal cations include, but are not limited, Na⁺, K⁺, Li⁺, and the like. Examples of suitable alkaline earth metal cations include, but are not limited to, Ca²⁺, Mg²⁺, and the like. Examples of suitable transition metal cations include, but are not limited, Fe³⁺, Zn²⁺, and the like.

Thiol reducing agents contemplated for the present invention include, but are not limited to, dithiothreitol (DTT), dithioerythritol (DTE), cysteine, N-acetylcysteine, 2-mercaptoethanol, methyl thioglycolate, 3-mercapto-1,2-propanediol (monothioglycerol), 3-mercaptopropionic acid, thioglycolic acid, trithioglycerol (1,2,3-trimercaptopropane), 1,2-dithioglycerol (dimercaprol), glutathione, dithiobutylamine, thioacetic acid, meso-2,3-dimercaptosuccinic acid or 2,3-dimercaptopropane-1-sulfonic acid.

The concentration of the antioxidant may be at least about 0.5 mg/ml, at least about 1 mg/ml, at least about 2 mg/ml, at least about 3 mg/ml, at least about 4 mg/ml, at least about 5 mg/ml, at least about 6 mg/ml, at least about 7 mg/ml, at least about 8 mg/ml, at least about 9 mg/ml, at least about 10 mg/ml, at least about 11 mg/ml, at least about 12 mg/ml, at least about 13 mg/ml, at least about 14 mg/ml, at least about 15 mg/ml, at least about 16 mg/ml, at least about 17 mg/ml, at least about 18 mg/ml, at least about 19 mg/ml, at least about 20 mg/ml, at least about 21 mg/ml, at least about 22 mg/ml, at least about 23 mg/ml, at least about 24 mg/ml, at least about 25 mg/ml, at least about 26 mg/ml, at least about 27 mg/ml, at least about 28 mg/ml, at least about 29 mg/ml, at least about 30 mg/ml, at least about 31 mg/ml, at least about 32 mg/ml, at least about 33 mg/ml, at least about 34 mg/ml, at least about 35 mg/ml, at least about 36 mg/ml, at least about 37 mg/ml, at least about 38 mg/ml, at least about 39 mg/ml, at least about 40 mg/ml, at least about 41 mg/ml, at least about 42 mg/ml, at least about 43 mg/ml, at least about 44 mg/ml, at least about 45 mg/ml, at least about 46 mg/ml, at least about 47 mg/ml, at least about 48 mg/ml, at least about 49 mg/ml, at least about 50 mg/ml, at least about 55 mg/ml, at least about 60 mg/ml, at least about 65 mg/ml, at least about 70 mg/ml, at least about 75 mg/ml, at least about 80 mg/ml, at least about 85 mg/ml, at least about 90 mg/ml, at least about 95 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml or at least about 200 mg/ml. Advantageously, the concentration is at least about 5 mg/ml, at least about 10 mg/ml or at least about 20 mg/ml.

In another embodiment, the reducing agent may advantageously be sodium thioglycolate or monothioglycerol. The reducing agent may be thioglycolic acid, a derivative thereof or a salt thereof, such as calcium thioglycolate, sodium thioglycolate or ammonium thioglycolate.

The concentration of the reducing agent may be about 0.02 mg/ml, about 0.03 mg/ml, about mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.13 mg/ml, about mg/ml, about 0.14 mg/ml, about 0.15 mg/ml, about 0.16 mg/ml, about 0.17 mg/ml, about 0.18 mg/ml, about 0.19 mg/ml, about 0.2 mg/ml, about 0.21 mg/ml, about 0.22 mg/ml, about 0.23 mg/ml, about mg/ml, about 0.24 mg/ml, about 0.25 mg/ml, about 0.26 mg/ml, about 0.27 mg/ml, about 0.28 mg/ml, about 0.29 mg/ml, about 0.3 mg/ml, 0.31 mg/ml, about 0.32 mg/ml, about 0.33 mg/ml, about mg/ml, about 0.34 mg/ml, about 0.35 mg/ml, about 0.36 mg/ml, about 0.37 mg/ml, about 0.38 mg/ml, about 0.39 mg/ml, about 0.4 mg/ml, about 0.41 mg/ml, about 0.42 mg/ml, about 0.43 mg/ml, about mg/ml, about 0.44 mg/ml, about 0.45 mg/ml, about 0.46 mg/ml, about 0.47 mg/ml, about 0.48 mg/ml, about 0.49 mg/ml or about 0.5 mg/ml. Advantageously, the concentration is about 0.2 mg/ml.

The present invention also relates to methods for stabilizing protein vaccines which may comprise adding a detergent.

In one embodiment, the detergent may advantageously be a span, a tween, and/or a Triton (such as, for example but not limited to, Triton X-100, Triton N-101. Triton 720 and/or Triton X-200). Any nonionic surfactants having as a hydrophilic polyethylene oxide group and a hydrocarbon lipophilic or hydrophobic group may be contemplated for the present invention. Any pluronic detergents which may comprise triblock copolymers of ethylene oxide and propylene oxide are also contemplated for the present invention. The concentration of the antioxidant may be at least about 0.005% (v/v), at least about 0.01% (v/v), at least about 0.02% (v/v), at least about 0.03% (v/v), at least about 0.04% (v/v), at least about 0.05% (v/v), at least about 0.06% (v/v), at least about 0.07% (v/v), at least about 0.08% (v/v), at least about 0.09% (v/v), at least about 0.1% (v/v), at least about 0.11% (v/v), at least about 0.12% (v/v), at least about 0.13% (v/v), at least about 0.14% (v/v), at least about 0.15% (v/v), at least about 0.16% (v/v), at least about 0.17% (v/v), at least about 0.18% (v/v), at least about 0.19% (v/v), at least about 0.2% (v/v), at least about 0.21% (v/v), at least about 0.22% (v/v), at least about 0.23% (v/v), at least about 0.24% (v/v), at least about 0.25% (v/v), at least about 0.26% (v/v), at least about 0.27% (v/v), at least about 0.28% (v/v), at least about 0.29% (v/v), at least about 0.3% (v/v), at least about 0.31% (v/v), at least about 0.32% (v/v), at least about 0.33% (v/v), at least about 0.34% (v/v), at least about 0.35% (v/v), at least about 0.36% (v/v), at least about 0.37% (v/v), at least about 0.38% (v/v), at least about 0.39% (v/v), at least about 0.40% (v/v), at least about 0.41% (v/v), at least about 0.42% (v/v), at least about 0.43% (v/v), at least about 0.44% (v/v), at least about 0.45% (v/v), at least about 0.46% (v/v), at least about 0.47% (v/v), at least about 0.48% (v/v), at least about 0.49% (v/v), at least about 0.5% (v/v), at least about 0.55% (v/v), at least about 0.6% (v/v), at least about 0.65% (v/v), at least about 0.7% (v/v), at least about 0.75% (v/v), at least about 0.8% (v/v), at least about 0.85% (v/v), at least about 0.9% (v/v), at least about 0.95% (v/v), at least about 1% (v/v), at least about 1.1% (v/v), at least about 1.2% (v/v), at least about 1.3% (v/v), at least about 1.4 g/ml, at least about 1.5% (v/v), at least about 1.6% (v/v), at least about 1.7% (v/v), at least about 1.8% (v/v), at least about 1.9% (v/v) or at least about 2% (v/v). Advantageously, the concentration is at least about 0.05% (v/v), at least about 0.1% (v/v) or at least about 0.2% (v/v).

The effectiveness of the present invention may be tested in several ways. A variety of analytical techniques are employed to detect, monitor and characterize the chemical degradation of protein molecules (Pharm Biotechnol. 2002; 13:1-25). For example, sodium dodecyl sulfate-polyacrylamide gel electrophoresis (SDS-PAGE) under non-reducing conditions can detect large changes in protein mass and disulfide cross-links. Reverse-phase and ion exchange chromatography methods are useful in determining oxidation and deamidation, respectively. The application of mass spectrometry to the field of protein chemistry has proven to be invaluable in the detection of chemical changes in protein molecules (Free Radical Biology & Medicine. 2006; 41:1507-1520 and Protein Science. 2000; 9:2260-2268). Peptide mapping combined with mass spectrometry is commonly employed in the pharmaceutical industry to detect and characterize chemical modifications of specific amino acid residues. The protein is first digested with one or more enzymes to produce a specific set of peptides based on the cleavage sites in the primary sequence. These peptides can then be analyzed by mass spectrometry directly (i.e. matrix-assisted laser desorption ionization-time of flight mass spectrometry (MALDI-TOF) or after chromatographic separation (i.e. liquid-chromatography-mass spectrometry, LC-MS). Changes in the mass to charge ratio (m/z) of the peptides can be indicative of a chemical modification, which can be further explored by other analytical techniques such as tandem mass spectrometry (MS/MS) (Biotechniques. 2006; 40:790-798).

The rHA can be formulated and packaged, alone or in combination with other influenza antigens, using methods and materials known to those skilled in the art for influenza vaccines. In a preferred embodiment, HA proteins from two A strains and one B strain are combined to form a multivalent vaccine.

In a particularly preferred embodiment, the HAs are combined with an adjuvant, in an amount effective to enhance the immunogenic response against the HA proteins. At this time, the only adjuvant widely used in humans has been alum (aluminum phosphate or aluminum hydroxide). Saponin and its purified component Quil A, Freund's complete adjuvant and other adjuvants used in research and veterinary applications have toxicities which limit their potential use in human vaccines. However, new chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the protein within a proteoliposome as described by Miller et al., J. Exp. Med. 176:1739-1744 (1992) and incorporated by reference herein, and encapasulation of the protein in lipid vesicles such as NOVASOME™ lipid vesicles (Micro Vescular Systems, Inc., Nashua, N.H.) should also be useful.

In the preferred embodiment, the vaccine is packaged in a single dosage for immunization by parenteral (i.e., intramuscular, intradermal or subcutaneous) administration or nasopharyngeal (i.e., intranasal) administration. The effective dosage is determined as described in the following examples. The carrier is usually water or a buffered saline, with or without a preservative. The antigen may be lyophilized for resuspension at the time of administration or in solution.

The carrier may also be a polymeric delayed release system. Synthetic polymers are particularly useful in the formulation of a vaccine to effect the controlled release of antigens. An early example of this was the polymerization of methyl methacrylate into spheres having diameters less than one micron to form so-called nano particles, reported by Kreuter, J., Microcapsules and Nanoparticles in Medicine and Pharmacology, M. Donbrow (Ed). CRC Press, p. 125-148. The antibody response as well as the protection against infection with influenza virus was significantly better than when antigen was administered in combination with alumium hydroxide. Experiments with other particles have demonstrated that the adjuvant effect of these polymers depends on particle size and hydrophobicity.

Microencapsulation has been applied to the injection of microencapsulated pharmaceuticals to give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the antigens are all factors that must be considered. Examples of useful polymers are chitosans, polycarbonates, polyesters, polyurethanes, polyorthoesters and polyamides, particularly those that are biodegradable.

A frequent choice of a carrier for pharmaceuticals and more recently for antigens may be poly (D,L-lactide-co-glycolide) (PLGA). This is a biodegradable polymer that has a long history of medical use in erodible sutures, bone plates and other temporary prostheses, where it has not exhibited any toxicity. A wide variety of pharmaceuticals including peptides and antigens have been formulated into PLGA microcapsules. A body of data has accumulated on the adaptation of PLGA for the controlled release of antigen, for example, as reviewed by Eldridge, J. H., et al. Current Topics in Microbiology and Immunology. 1989, 146: 59-66. The entrapment of antigens in PLGA microspheres of 1 to 10 microns in diameter has been shown to have a remarkable adjuvant effect when administered orally. The PLGA microencapsulation process uses a phase separation of a water-in-oil emulsion. The compound of interest is prepared as an aqueous solution and the PLGA is dissolved in a suitable organic solvents such as methylene chloride and ethyl acetate. These two immiscible solutions are co-emulsified by high-speed stirring. A non-solvent for the polymer is then added, causing precipitation of the polymer around the aqueous droplets to form embryonic microcapsules. The microcapsules are collected, and stabilized with one of an assortment of agents (polyvinyl alcohol (PVA), gelatin, alginates, polyvinylpyrrolidone (PVP), methyl cellulose) and the solvent removed by either drying in vacuum or solvent extraction.

The compositions of the invention may be injectable suspensions, solutions, sprays, lyophilized powders, syrups, elixirs and the like. Any suitable form of composition may be used. To prepare such a composition, a protein formulation of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al, (2002) J. Leuk. Biol. 71(3): 538-44; Ahmad-Nejad, P. et al (2002) Eur. J. Immunol. 32(7): 1958-68; poly IC or poly AU acids, polyarginine with or without CpG (also known in the art as IC31; see Schellack, C. et al (2003) Proceedings of the 34th Annual Meeting of the German Society of Immunology; Lingnau, K. et al (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al (2004) 22(13-14): 1791-8), and the CCR5 inhibitor CMPD167 (see Veazey, R. S. et al (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al (2001) J. Immunol. 167(6): 3398-405), polyphosphazenes (Allcock, H. R. (1998) App. Organometallic Chem. 12(10-11): 659-666; Payne, L. G. et al (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD4OL (ADX40; see, for example, WO03/063899), and the CD1a ligand of natural killer cells (also known as CRONY or α-galactosyl ceramide; see Green, T. D. et al, (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the antigens of the invention or on separate expression vectors.

The immunogenic compositions can be designed to introduce the rHAs to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

The methods of the invention can be appropriately applied to prevent diseases as prophylactic vaccination or treat diseases as therapeutic vaccination.

The vaccines of the present invention can be administered to an animal either alone or as part of an immunological composition.

Beyond the human vaccines described, the method of the invention can be used to immunize animal stocks. The term animal means all animals including humans. Examples of animals include humans, cows, dogs, cats, goats, sheep, horses, pigs, turkeys, ducks, chickens, etc. Since the immune systems of all vertebrates operate similarly, the applications described can be implemented in all vertebrate systems.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Mechanism of H3 rHA Potency Loss—Cysteine Mutagenesis

This Example was designed to determine the importance of specific Cys residues on potency loss for H3 rHA. The last Cys residue in the HA sequence was associated with potency loss in H3 Perth rHA and H3 Victoria rHA. However, the HA proteins from H3 human influenza strains also contain two additional Cys residues in the transmembrane domain (TM) domain compared to H1 and B human influenza strains (FIG. 2). The Cys residues in the TM of HA proteins are not conserved among the human influenza strains and two additional residues are in the TM domain of the H3N2 strains.

In this Example, cysteine residues in rHA H3 Perth were replaced with Serine or Alanine. The three constructs of H3 A/Perth/16/2009 rHA prepared for this Example are listed in Table 1.

TABLE 1
rHA Variant Proteins in the Cysteine Mutagenesis Study

| | rHA | | Location of |
|---|---|---|---|
| Construct # | Protein | Mutations | Mutations |
| 1 | H3 Perth | C524S, C528A, C539A, | TM (CT) |
| | | (C546A, C549A) | |
| 2 | H3 Perth | C539A, (C546A, C549A) | TM (CT) |
| 3 | H3 Perth | C524S, C528A | TM |

The constructs include mutations in the transmembrane domain (TM) and the cytoplasmic tail (CT). The cysteine residues of the TM and CT domains in the HA monomer are thought to be in close proximity to each other in the homotrimer, and potentially in rosette structures of rHA, and may readily form disulfide bonded rHA multimers as a result. In addition, the cysteine residues in the CT domain are acylated in insect cells, and this modification may affect stability of the protein. All five cysteine residues in the TM and CT domains have been mutated in construct 1, while the three cysteine residues in the CT domain have been mutated in construct 2. The two additional cysteine residues unique to H3 HA proteins in the TM domain have been mutated in construct 3 to residues commonly observed in HA proteins derived from both human and animal origins.

The Cys residues were mutated to Alanine in all positions except 524.

The selected testing is provided in Table 2.

TABLE 2

| Product Attribute | Method |
|---|---|
| Starting Yield | SRID potency |
| Purity | SDS-PAGE profile |
| Final Yield | BCA adjusted for purity |
| Stability: 28-day Relative Potency (RP) | SRID potency |
| Aggregation/Cross-linking | SDS-PAGE |

### Example 2

### Effect of Cysteine Residues on the Stability of rHA

Based on stability data for recombinant hemagglutinins in Flublok™, disulfide mediated cross-linking increases with bulk age and is associated with potency loss. In general, the H3 rHA proteins are considered less stable than H1 and B rHA proteins based on real time stability data for manufacturing batches produced between 2007 and 2011 (FIG. 3). Due to its rapid potency loss in the SRID assay (FIG. 3), H3/Perth/16/2009 (H3/Perth) rHA was used as a model protein to develop methods to improve stability and to investigate mechanisms of potency loss. The stability of this protein was improved and its non-cross-linked state preserved through the addition of citrate and sodium thioglycolate, a reductant, to the existing formulation. For these reasons, cysteine residues are thought to play an important role in rHA stability.

Three different plasmid DNA constructs of H3/Perth rHA were prepared (Table 1 of Example 1). The constructs of H3/Perth rHA contain point mutations at coding regions for specific cysteine residues replacing them with either a serine or an alanine. Specifically, cysteine residues in the transmembrane domain (TM) and cytoplasmic tail (CT) of the protein were targeted.

Constructs 1 & 2: These constructs include mutations in the transmembrane domain (TM), the cytoplasmic tail (CT). The cysteine residues of the TM and CT domains in the HA monomer are thought to be in close proximity to each other in the homotrimer and potentially in rosette structures of rHA, and may readily form cross-links as a result. In addition, the cysteine residues in the CT domain may be acylated in insect cells and this modification could affect stability of the protein. All five cysteine residues in the TM and CT domains are mutated in construct 1, while the three cysteine residues in the CT domain are mutated in the construct 2.

Construct 3: HA proteins from H3 human influenza strains contain two additional cysteine residues in the TM domain compared to H1 and B human influenza strains (FIG. 2). These two additional cysteine residues in H3/Perth rHA (C524 and C528) are mutated in construct 3 to residues commonly observed in HA proteins derived from both human and animal origins.

Examples 1 and 2 include three different plasmid DNA constructs encoding variants of the H3 A/Perth/16/2009 (H3 Perth) rHA protein. The plasmid DNA constructs are prepared by polymerase chain reactions (PCRs). Amino acid residue changes are introduced by two complementary site directed mutagenesis (SDM) primers which contain sense mutation of the nucleotide(s). See Table 3, below, for the primers used for SDM. The transfer vector pPSC12 LIC containing the wild-type HA gene for the H3 Perth rHA protein is used as a template in the PCR for constructs 2 and 3. The mutagenized construct 2 plasmid DNA is used as a template in the PCR for construct 1.

TABLE 3
Primers used to Generate H3/Perth rHA and B/Brisbane rHA Variant
Proteins

| | | | |
|---|---|---|---|
| Construct | | | |
| # | Primer # | Mutations | Primer sequence, 5′-3′ |

H3/Perth/16/20009

| | | | |
|---|---|---|---|
| 1 | 3928 | C539A.C546A.C549A | CCTTTGCCATATCAgcTTTTTTGCTTgcTGTTGCTTTGTTGGGG |
| | (forward) | | |
| | 3929 | C524A.C528A | CCCCAACAAAGCAACAgcAAGCAAAAAAgcTGATATGGCAAAGG |
| | (reverse) | | |
| 2 | 3891 | C539A.C546A.C549A | GGGGTTCATCATGTGGGCCgcCCAAAAAGGCAACATTAGGgc |
| | (forward) | | CAACATTgcCATTTAAGTAAGTACCG |
| | 3892 | | CGGTACTTACTTAAATGgcAATGTTGgcCCTAATGTTGCCTTTTT |
| | (reverse) | | GGgcGGCCCACATGATGAACCCC |
| 3 | 3889 | C524S.C528A | CCTTTGCCATATCATcTTTTTTGCTTgcTGTTGCTTTGTTGGGG |
| | (forward) | | |
| | 3890 | | CCCCAACAAAGCAACAgcAAGCAAAAAAgATGATATGGCAAAGG |
| | (reverse) | | |

Bold and lowercase type denotes the nucleotides designed to introduce mutations in the rHA.

The PCR amplified products include the synthesized, mutagenized plasmid. The PCR reactions are treated with the restriction endonuclease DpnI, an enzyme which cleaves its recognition site only when it is methylated. Treatment with DpnI results in digestion of the template plasmid DNA, while the PCR synthesized plasmid DNA remains circularized. The DpnI treated PCR reaction is then used to transform E. coli.

Five to 10 plasmid DNA samples are submitted for sequencing using HA specific primers to verify SDM of the targeted amino acid residue(s) only. The sequences containing the cloned rHA genes and the flanking regions of the transfer plasmid are sequenced using primers spaced approximately every 300 nucleotides. The sequencing reactions are carried out by MWG-Operon (Huntsville, Ala.). The resulting sequence data are assembled using SeqMan software from DNASTAR, Inc. (Madison, Wis.) or Vector NTI (Invitrogen). Data from individual sequencing runs for each clone are compiled into a single contiguous sequence which is then compared to the reference sequence (wild-type) to ensure that the correct protein is encoded by the clone and the desired mutation has been introduced.

TABLE 4
Acceptance Criteria for Cloning

| Process Step | Product Attribute | Method | Criteria |
|---|---|---|---|
| Clone Selection | Identity | Sequence | Site specific mutation(s) |
| | | Analysis | observed. The rest of |
| | | | HA sequence is |
| | | | confirmed to be |
| | | | wild-type. |

Baculovirus Generation and Scale-Up. The recombinant baculovirus is prepared by homologous recombination and transfection into insect cells. AcMNPV baculovirus DNA from the Master Virus Bank is digested with Bsu 361 to remove the polyhedrin gene and a portion of open reading frame (ORF) 1629. The linearized parental AcMNPV DNA and the pPSC12 LIC transfer plasmid DNA containing the rHA gene of interest are combined and added to the liposome transfection reagent, a 1:2 molar ratio of Dimethyldidecylammonium Bromide (DDAB) and 1,2-Dioleoyl-sn-glycero-3-phosphoethanolamine (DOPE). After incubating at room temperature, the transfection solution is added to a culture of expresSF+ cells that are freshly seeded in a 125 mL shake flask. The transfection is incubated for ˜5 days at 22-28° C. with shaking. Once the cell diameter is >21 μm, the transfection is harvested by centrifugation and the supernatant isolated for plaque purification.

The viral supernatant from the transfection is used to infect a monolayer of insect cells in order to purify and isolate recombinant plaques for further scale-up. Monolayers of SF+ cells in early to mid-log phase are inoculated with serial dilutions of the transfection supernatant. A 2×PSFM/Agarose overlay is applied to the plates. After 5-10 days at 26-28° C., well isolated recombinant plaques are identified by microscopic evaluation under low magnification and by comparison with a control of wild-type baculovirus plaques expressing the polyhedron gene. Recombinant plaques are harvested from the agarose and transferred to a culture of cells for scale-up to virus passage 1 (P1). The transfection and recombinant plaque isolation steps are evaluated according to the acceptance criteria provided in Table 5 below.

TABLE 5
Acceptance Criteria for Transfection and Recombinant Baculovirus
Identification

| Process Step | Product Attribute | Method | Criteria |
|---|---|---|---|
| Transfection | Cell Diameter | Vi-Cell | >21 μm |
| Plaque Isolation | Morphology | Microscopic | Wild-type |
| | | | baculovirus |
| | | | morphology |
| | | | negative |

The plaque-purified recombinant baculoviruses are amplified into passage 3 (P3) Working Virus Banks (WVB) by propagation of virus passage 1 (P1) through passage 3 in SF+ cells under serum-free conditions. The isolated recombinant plaque is used to infect a culture of SF+ cells in early to mid-log phase in a 125 mL shake flask. The infected culture is incubated for at least 5 days at 26-28° C. with shaking and is harvested by centrifugation after criteria for cell density and viability are met (cell density>20 μm; cell viability<80%). The supernatant containing the P1 virus is used to prepare passage 2 (P2) virus. The DNA from an aliquot of the P1 virus is isolated and tested for the correct gene product using PCR. See Table 6 below.

For Passages 2 and 3, SF+ cell cultures are seeded at a density of 1.0×10⁶ cells/mL and are incubated at 26-28° C. for 18-24 hours to reach an infection cell density of 1.3−1.7×10⁶ cells/mL prior to infection with P1 or P2 virus supernantants. The infected culture is incubated at 26-28° C. with shaking and harvested by centrifugation after 24 hours and after criteria for P2 (cell density increases; cell viability 40-70%) and P3 (cell density increases; cell viability<70%) are met. The P3 virus in the supernatant is tested to determine its titer using the virus titration assay and to confirm HA gene insertion. See Table 7 below. The cell pellets obtained from harvesting the cultures in P2 and P3 are resuspended in 1×PBS and analyzed by SDS-PAGE gel electrophoresis and western blot to confirm the expression of the rHA protein. See Table 6 below.

The working virus bank scale-up is evaluated according to the acceptance criteria provided in Table 6 below.

TABLE 6
Acceptance Criteria for the Working Virus Bank Scale-Up

| Process | Product | | |
|---|---|---|---|
| Step | Attribute | Method | Criteria |
| Passage 1 | Identity rHA | PCR | ~2.5 kb gene product |
| | Gene Product | | for HA observed |
| Passage 2 | Identity rHA | SDS-PAGE/ | 62 kD protein band/ |
| | protein | Western Blot | 62 kD immunoreactive band |
| Passage 3 | Identity rHA | SDS-PAGE/ | 62 kD protein band/ |
| | protein | Western Blot | 62 kD immunoreactive band |
| | Identity rHA | PCR | ~2.5 kb gene product |
| | Gene Product | | for HA observed |

The P3 Working Virus Bank is stored frozen in liquid nitrogen for at least 2 years after the addition of DMSO (10%) to the viral supernatant. Alternatively, the P3 working virus bank is stored at 2-8C for up to 8 weeks.

P5 Scale-up and Fermentation. The Fermentation is infected with P5 virus generated by further propagating the P3 Working Virus Bank. For the P4 and P5 virus, SF+ cell cultures are seeded at a density of 1.0×10⁶ cells/mL in shake flasks and are incubated at 26-28° C. for 18-24 hours to reach an infection cell density of 1.3−1.7×10⁶ cells/mL. The P4 culture is infected with the P3 working virus bank, and the P5 is infected with the P4 viral supernatant. P4 and P5 viral supernatants are isolated by centrifugation of the culture after meeting criteria for P4 (cell viability between 35% and 70%) and P5 (cell viability between 35% and 70%) virus. The resulting P4 virus and P5 virus are stored at 2-8° C. for 8 and 4 weeks, respectively. Cell pellets obtained from harvesting the cultures in P4 and P5 are resuspended in 1×PBS and analyzed by SDS-PAGE gel electrophoresis/Western blot to confirm the expression of the rHA protein.

The working virus bank scale-up to P5 and the fermentation are evaluated according to the acceptance criteria provided in Table 7 below.

TABLE 7
Acceptance Criteria for the P5 Virus Scale-up and Fermentation

| Process | Product | | |
|---|---|---|---|
| Step | Attribute | Method | Criteria |
| Passage 4 | Identity rHA | SDS-PAGE Western | 62 kD immunoreactive |
| | protein | Blot | band |
| Passage 5 | Identity rHA | SDS-PAGE Western | 62 kD immunoreactive |
| | protein | Blot | band |
| Infection | Cell Viability | Vi-Cell analysis | Viability drop |
| Harvest | Free of | Microscopy | Absence of bacterial |
| | contamination | | or fungal growth |
| | Cell Viability | Vi-Cell analysis | 40-80% |
| | Identity rHA | SDS-PAGE Western | 62 kD immunoreactive |
| | protein | Blot | band |

The wild-type and variant rHA proteins in this Example are produced in 15 L bioreactors having a working volume of 10 L. A culture of SF+ cells is seeded with SF+ cells in PSFM media. The culture is maintained at specified agitation rate at 26-28° C. Bioreactors are equipped with an air overlay, and a specified dissolved oxygen concentration. When the culture reaches a pre-determined density with sufficient viability, it is infected with the P5 working virus bank. The fermentation is sampled and examined by light microscopy at 400× magnification for bacterial or fungal contamination. The fermentation is harvested when cell viability is within 40%-80%.

The fermentation is harvested by centrifugation. The 10 L fermentation is pumped into sterile 1 L bottles in ˜1 L aliquots and centrifugation using a Sorvall RC3C swinging bucket centrifuge at 2-8° C. The cells are pelleted and collected while the supernatant containing spent medium from the fermentation is discarded. The pellets are either purified immediately or stored frozen at ≦20° C. until further purification.

Protein Purification. Purification of the rHA protein is done at the 4 L or 10 L scale using cell pellets obtained from ˜4 L or ˜10 L of fermentation, respectively. Cell pellets are purified immediately after harvesting or after storage at −20° C. Frozen pellets are completely thawed at 2-8° C. prior to purification. The small scale operations in this Example are described for each purification step below. The purification involves the following steps: Extraction, IEX Chromatography, HIC Chromatography, Q-Filtration, Ultrafiltration. Formulation and Final Filtration. Criterion for assessing the process step and/or the product (process intermediate) quality are provided for each unit operation.

Extraction. In this step, the rHA protein is solubilized from the cell membrane using Triton X-100 surfactant and released into a buffer for further purification.

This step is performed at 2-8° C. Pre-chilled (2-8° C.) Triton® X-100 extraction buffer is added to the cell pellet obtained by centrifugation and mixed on a stir plate with a stir bar. After the minimum mixing period, an aliquot of the suspension (Crude Extract) is sampled and centrifuged. The supernatant is isolated and tested to determine the starting yield. The resulting Crude Extract is immediately processed without hold.

TABLE 8
Process Requirements for Purification of rHA - Extraction

| | Process | | |
|---|---|---|---|
| Process Step | Requirement | Method | Criteria |
| Extraction (5) | Starting Yield | SRID | ≧70% of the wild-type rHA |

Depth Filtration. Depth Filtration is performed to remove cell debris and suspended solids and reduce turbidity. The filter containing cell debris and particulates is discarded and the rHA is recovered in the filtrate stream.

The filtration step uses a single lenticular depth filter washed with PUW and pre-equilibrated with rHA specific extraction buffer. The filtration is performed at 22-28° C. while mixing of the Crude Extract continues to prevent settling of the cell pellet debris during filter loading. The process intermediate, Depth Filtrate, is immediately processed in the next step.

IEX Chromatography. The Ion Exchange (IEX) Chromatography step uses a SP BB cation exchange column to capture and concentrate the rHA protein in the Depth Filtrate. Contaminant proteins that do not bind to the column are removed in the flow through and the washes.

The IEX column is equilibrated until pH and conductivity requirements are met. The IEX-Load is pumped onto the equilibrated IEX column. After loading and prior to elution, the column is washed using rHA specific buffers to remove additional/residual contaminants. The rHA is eluted from the column with sodium chloride under isocratic conditions and the UV280 absorbance peak collected. An aliquot of the absorbance peak is collected for testing to confirm the presence of a ˜65 kD protein in the IEX-Eluate and yield. The IEX-Eluate is collected and further processed in <24 hours.

TABLE 9
Process Requirements and Product Attributes for Purification of rHA-IEX
Chromatography

| | Process | | |
|---|---|---|---|
| | Requirement/ | | |
| | Product | | |
| Process Step | Attribute | Method | Criteria |
| IEX | Yield | Total Protein (BCA) | ≧60% of |
| Chromatography | | adjusted for | wild-type rHA |
| | | SDS-PAGE purity | |
| | Elution Profile | UV₂₈₀ Absorbance | Overlays with |
| | | | wild-type rHA |
| | Identity | SDS-PAGE | presence of a |
| | | | ~65 kD protein |

HIC Chromatography. The Hydrophobic Interaction Chromatography (HIC) step uses a Phenyl HP chromatography column to purify the rHA protein in the IEX-Eluate.

The HIC column is washed with water and equilibrated with equilibration buffer until pH and conductivity requirements are met. The IEX-Eluate is adjusted for column loading by diluting with an equal volume of detergent free buffer and CHAPS surfactant is added using a 10% stock solution of the surfactant. After loading, the column is washed with rHA specific buffers and protein contaminants in the flow-through and the washes are discarded. The rHA is eluted with elution buffer and the entire UV280 absorbing peak is collected in fractions.

The elution fractions are stored until the rHA content is confirmed by SDS-PAGE, and the elution fractions containing rHA are then pooled. The resulting rHA pool is designated the HIC-Eluate. The HIC-Eluate is collected, pooled, and further processed in <24 hours.

TABLE 10
Process Requirements and Product Attributes for Purification of rHA-HIC
chromatography

| | Product | | |
|---|---|---|---|
| Process Step | Attribute | Method | Criteria |
| HIC | Yield | Total Protein | ≧60% of wild-type rHA |
| Chromatography | | (BCA) adjusted | |
| (8) | | for SDS-PAGE | |
| | | purity | |
| | Elution | UV₂₈₀ | Overlays with wild-type |
| | Profile | Absorbance | rHA |
| | Identity | SDS-PAGE | presence of a ~65 kD |
| | | | protein |

Q Membrane Filtration. Q Membrane Filtration is performed using a Pall Mustang Q coin filter to remove DNA from the rHA.

Q membrane filtration is performed at 22-28° C., and the filter is sanitized, washed, and preconditioned for use. The capsule is equilibrated with a rHA specific buffer until pH and conductivity specifications are met. The HIC-Eluate from the previous step is conditioned for Q Membrane Filtration using a stock solution of 1M NaCl. The adjusted HIC-Eluate is referred to as the Q-Load. The Q-Load is filtered through the capsule via pump, and the UV absorbing material (280 nm) is collected. The Q capsule is washed with rHA buffer until the UV absorbance returns to baseline. The collected material, i.e., the filtrate and wash, is designated the Q-Filtrate. The Q-Filtrate is sampled for testing to determine the total protein concentration. The Q-Filtrate is processed immediately or stored at 2-8° C. for <24 hours until subsequent processing.

TABLE 11
Product Attribute for Purification of rHA-Q Filtration

| | Product | | |
|---|---|---|---|
| Process Step | Attribute | Method | Criteria |
| Q Filtration | Concentration | Total Protein | FIO |
| | | (BCA) | (expected ≧800 μg/mL) |

Ultrafiltration. Ultrafiltration for buffer exchange of the rHA protein is performed at 22-28° C. using a Pall Minimate Tangential Flow Filtration (TFF) capsule, a flat plate polyethersulfone (PES) membrane with a nominal molecular weight limit (NMWL) of 50 kD. Prior to use, the filter is flushed with PUW and equilibrated with buffer. The Q-Filtrate is recirculated through the system to further condition the membrane. After recirculation, a 10-fold minimum buffer exchange is performed in a constant volume mode using rHA buffer.

The Retentate obtained from diafiltration is weighed to determine the mass and is sampled for testing to determine the total protein concentration and total protein yield.

TABLE 12
Process Requirements and Product Attributes for Purification of rHA-
Ultrafiltration

| | Process | | |
|---|---|---|---|
| | Requirement/ | | |
| Process Step | Product Attribute | Method | Criteria |
| Ultrafiltration | Concentration | Total Protein (BCA) | ≧400 μg/mL |
| | Yield | Total Protein (BCA) | ≧60% of |
| | | | wild type yield |

Formulation and Final Filtration. In this Example, the total protein concentration of the purified rHA in the Retentate must be between 400-600 μg/mL. The Retentate may be further concentrated by TFF or diluted with diafiltration buffer to achieve this concentration, if necessary.

The formulation for the rHA proteins in this Example is 10 mM sodium phosphate, 150 mM sodium chloride, 0.005% Tween-20, pH6.8-7.2. To achieve this formulation, Tween-20 is added to the Retentate to a final concentration of 0.005% Tween-20 using a 10% Tween-20 stock solution. The resulting intermediate is the Formulated Retentate.

To generate the Monovalent Bulk rHA for testing in this Example, the Formulated Retentate is simultaneously filtered through a 0.2 μm filter and transferred from the formulation container into a bioprocess container for storage.

Storage and Stability. Testing of rHA Proteins. After formulation and fill are complete, BPCs containing wild-type and mutant rHA protein will be placed on 28 day accelerated stability at 22-28° C. The BCA, Trypsin Resistance, and Agglutination assays are performed on day 0. All other tests are performed on each timepoint. Testing is completed within +/−1 day of the target test day. Adjustments to the schedule may be made to accommodate laboratory schedules or experimental observations.

Acceptance Criteria. Stability data is assessed by comparing the purity, yield, stability profiles, aggregation profiles, and folding for the wild-type rHA proteins to those of the corresponding rHA variants. RP-HPLC analysis is for information only and any differences in the RP-HPLC profiles for wild-type and mutant rHA proteins are noted.

Purity is determined by SDS-PAGE.

Yield is determined by BCA adjusted for purity.

Stability is indicated by the results for potency as measured by SRID.

Aggregation and cross-linking is assessed from the reducing and non-reducing SDS-PAGE gels.

Proper folding is assessed by trypsin resistance and agglutination of red blood cells.

TABLE 13
Product Attributes for Purified Wild-type and Variant rHA Proteins

| Product Attribute | Method | Criteria |
|---|---|---|
| Purity | SDS-PAGE profile | ≧85% |
| Yield | BCA adjusted | ≧70% of wild-type |
| | for purity | |
| Stability: | SRID potency | 28-Day RP_{mutant rHA} |
| 28-day Relative | | ≧28-Day RP_{wild-type rHA} |
| Potency (RP) | | |
| Aggregation/ | SDS-PAGE | Aggregate band intensity of |
| Cross-linking | | mutant rHA < aggregate band |
| | | intensity of wild-type rHA |
| Trypsin Resistance | SDS-PAGE or | HA1 and HA2 observed |
| (proper folding) | Western Blot | |
| Agglutination | Red blood cell assay | Positive for agglutination of |
| (proper folding) | | red blood cells |
| RP-HPLC Profile | RP-HPLC | FIO |

### Example 3

### Cysteine Mutagenesis

Cloning—Three different constructs of the H3 A/Perth/16/2009 rHA protein were prepared for comparison with the wild-type H3 A/Perth/16/2009 rHA protein. In these constructs, specific cysteine residues in the transmembrane and cytoplasmic tail domains of the rHA protein were replaced. The mutations in these constructs are shown below.

TABLE 14

| rHA | | Construct |
|---|---|---|
| Protein | Mutations | Name |
| H3 Perth | C524S, C528A | 2 Cys TM |
| | | (H3) |
| H3 Perth | C524A, C528A, C539A, C546A, | 5Cys (H3) |
| | C549A | |
| H3 Perth | C539A, C546A, C549A | 3Cys (H3) |
| H3 Perth | None (Wild-type) | Wild-type |

The constructs, virus banks, and fermentations were prepared for the H3 rHA proteins. The H3 rHA proteins were purified and characterized according to the protocol of Example 2. The results for the H3 Perth rHAs are provided below.

Initial rHA Clone Screen—Small scale fermentations (300 mL) were prepared for the H3 rHA variants and the starting yield determined for comparison with the wild-type H3 rHA. All H3 rHA variants met yield criteria except for one.

TABLE 15

| | | Cell | Average | % of Wild-type Control |
|---|---|---|---|---|
| rHA | HPI @ | Viability | mg/Liter of | Criteria: ≧70% of wild- |
| A/Perth | Harvest | % | Fermentation | type |
| 2 Cys | 48 | 52.2 | 51.6 | 112.1 |
| 3 Cys | 48 | 57.4 | 58.8 | 127.8 |
| 5 Cys | 48 | 42 | 47.5 | 103.2 |
| Wild-type | 48 | 48.8 | 46 | 100 |
| 2 Cys | 66 | 21.7 | 58 | 98.7 |
| 3 Cys | 66 | 23.2 | 68.3 | 116.1 |
| 5 Cys | 66 | 21.6 | 60.9 | 103.6 |
| Wild-type | 66 | 19.5 | 58.8 | 100 |

Starting Yields—Three Cys H3 rHA variants were scaled-up (10 L) and purified (4 L scale) for comparison with the wild-type H3 rHA. At the 10 L fermentation scale, the starting yields for the three Cys variants are essentially the same or greater than that of the wild-type control and meet study criteria.

TABLE 16
H3/Perth

| | | | | | % of Wild- |
|---|---|---|---|---|---|
| | Cell | | SRID | Starting | type Control |
| | Viability | HPI @ | Potency | Yield | Criteria: ≧70% |
| rHA | % | Harvest | μg/mL | mg/LoF | of wild-type |
| Wild-Type | 45.9 | 55 | 85 | 34 | 100 |
| 3 Cys Mutant | 52.5 | 55 | 155 | 62 | 182 |
| 5 Cys Mutant | 48.9 | 55 | 102 | 40.8 | 120 |
| 2 Cys Mutant | 36.9 | 55 | 117 | 46.8 | 138 |

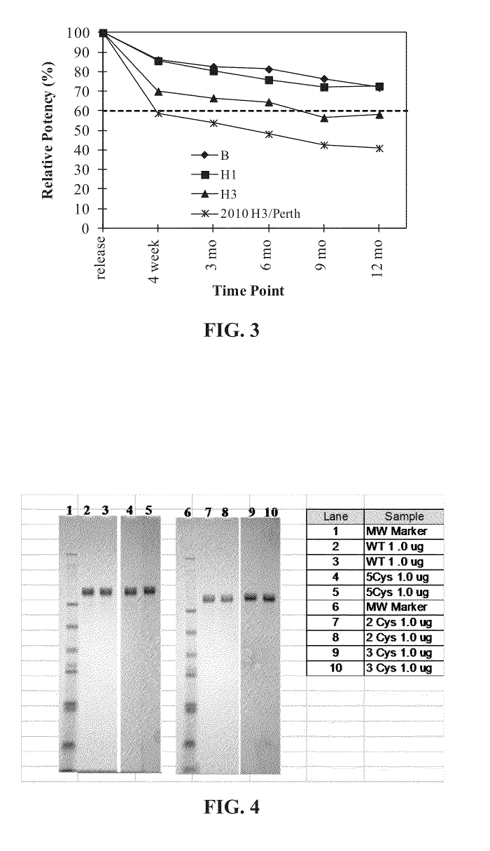

Purity—The purified H3 rHA proteins have a purity of 100% by reducing SDS-PAGE gel analysis using a 1 μg/lane loading. The study criterion for purity by SDS-PAGE is ≧85% (FIG. 4).

Final Yields—The final, purified yields for the three Cys H3 rHA variants are essentially the same or greater than that of the wild-type control.

TABLE 17

| | BCA | | | % of Wild- |
|---|---|---|---|---|
| | adjusted | | | type Control |
| | Purity | Total Weight | Yield | Criteria: ≧70% |
| rHA Protein | μg/mL | g | mg | of wild-type |
| H3 Perth wild-type | 465 | 37 | 17.2 | 100 |
| H3 Perth 5Cys | 497.25 | 56.9 | 28.3 | 164 |
| H3 Perth 3Cys | 474.448 | 50.3 | 23.9 | 139 |
| H3 Perth 2Cys | 503.87 | 78.5 | 39.6 | 230 |

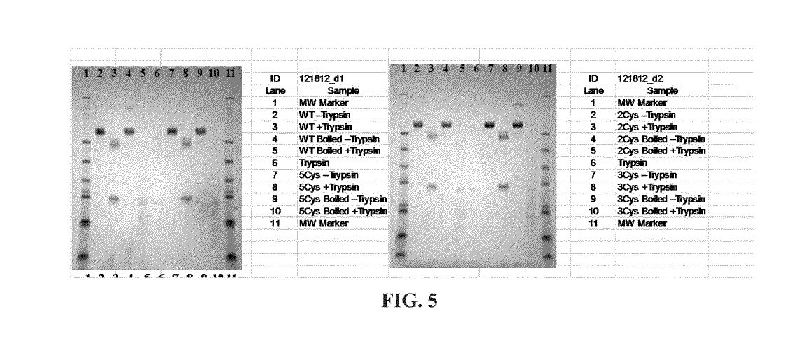

Trypsin Resistance—The wild-type H3 rHA and the Cys mutants have trypsin resistance indicating that the rHA proteins are properly folded and trimeric. All H3 rHAs met the study criteria for the assay, visible bands for HA1 and HA2 (FIG. 5).

Hemagglutination Assay—The wild-type H3 Perth rHA and the Cys mutants are positive for hemagglutination activity meeting the study criteria and indicating that the rHA proteins are properly folded. Study criteria: Positive for agglutination of red blood cells.

TABLE 18

| | | | | HA Activity |
|---|---|---|---|---|
| rHA | Clone | ID | Desc. | Units/μg |
| H3 Perth | D7403.2eQ | H3-12068 | W.T. | 160 |
| H3 Perth | D7735.1aQ | H3-12069 | 5 Cys | 40 |
| H3 Perth | D7713.5aQ | H3-12070 | 2 Cys | 240 |
| H3 Perth | D7734.3aQ | H3-12071 | 3 Cys | 40 |

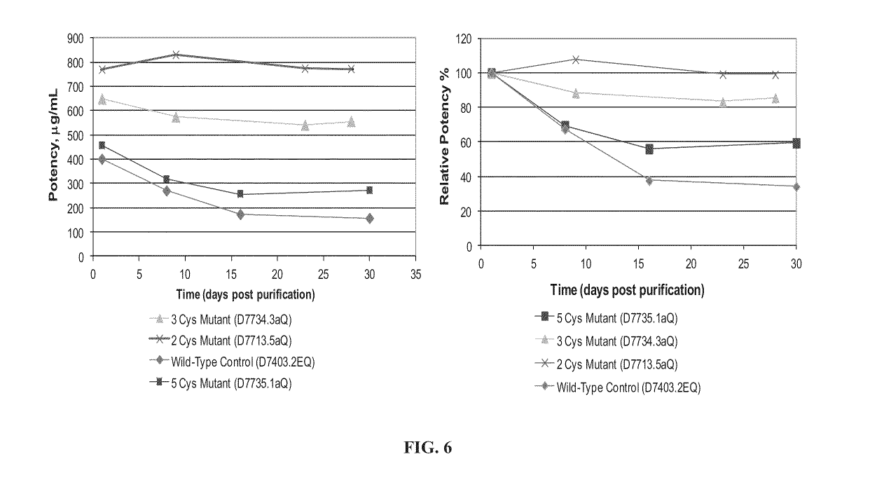

Potency by SRID—After 1 month at 25° C., the wild-type H3 rHA protein showed the greatest potency drop and stabilized at a relative potency of −40%. The relative potency for the 5Cys H3 rHA stabilized at −60%. The potency drop for the 3Cys H3 rHA was less than 20%, and the 2Cys H3 rHA shows no potency loss. All three Cys H3 rHA variants meet study requirements for relative potency (RP) on day 28. (FIG. 6)

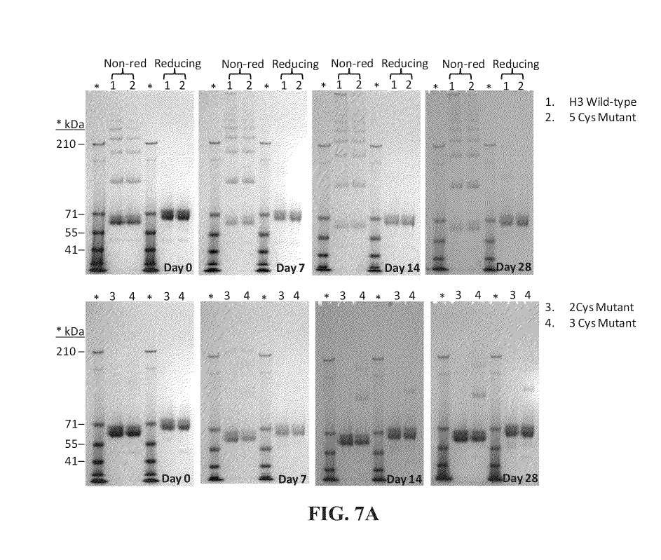

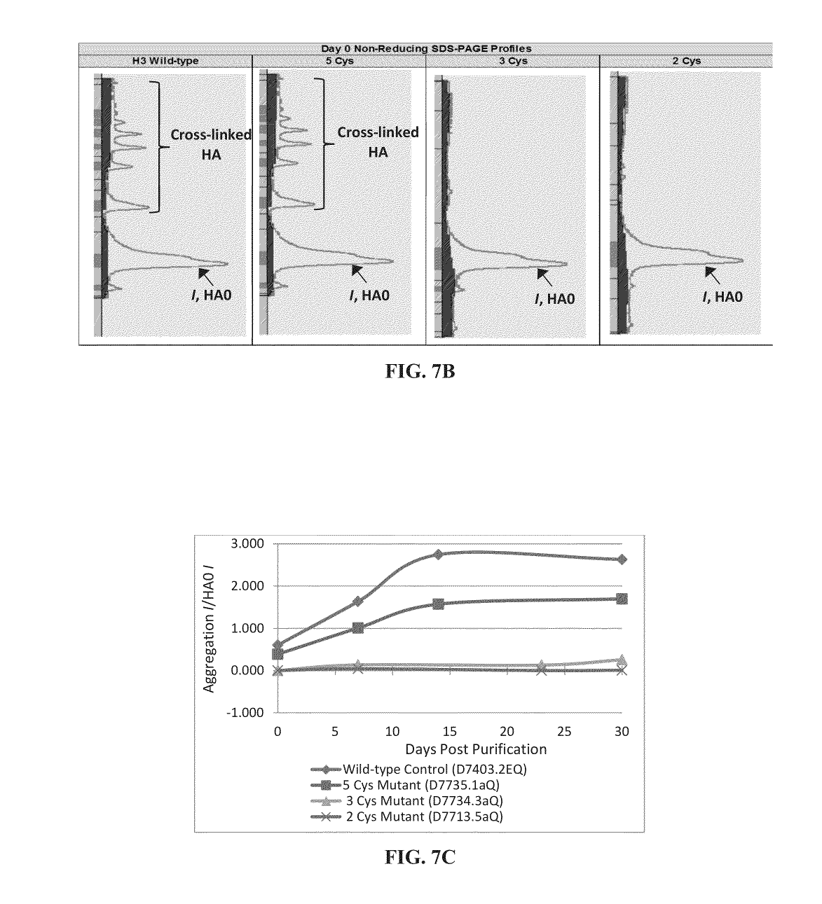

SDS-PAGE—The non-reducing and reducing SDS-PAGE profiles for the wild-type H3 rHA protein and the three different Cys variant rHAs is shown in FIG. 7A. On day 0, the non-reducing SDS-PAGE profile for the wild-type and 5 Cys mutant are comparable to each other, however, more rHA cross-linking is observed in the wild-type rHA compared to the 5 Cys mutant on all subsequent time points. The 3 Cys and 2 Cys mutants have little to no cross-linking on day 0, and increases slightly in the 3Cys mutant only. Study criteria, aggregate band intensity of mutant rHA<aggregate band intensity of wild-type rHA, were met.

The non-reducing SDS-PAGE gels were scanned and analyzed using molecular imaging software. The intensity profiles from the imaging analysis are shown in FIG. 7B for day 0 of the study.

Densitometry was performed on the non-reducing SDS-PAGE gels at each time point and for each H3 rHA protein. The band intensities for the monomeric rHA protein (HA0) and the higher cross-linked forms of the rHA protein (aggregation) were determined. A ratio of the aggregation intensity to the intensity of HA0 was plotted below for each H3 rHA. By this method, rHA cross-linking increases in the order 2Cys<3Cys<5Cys<Wild-type. (FIG. 7C)

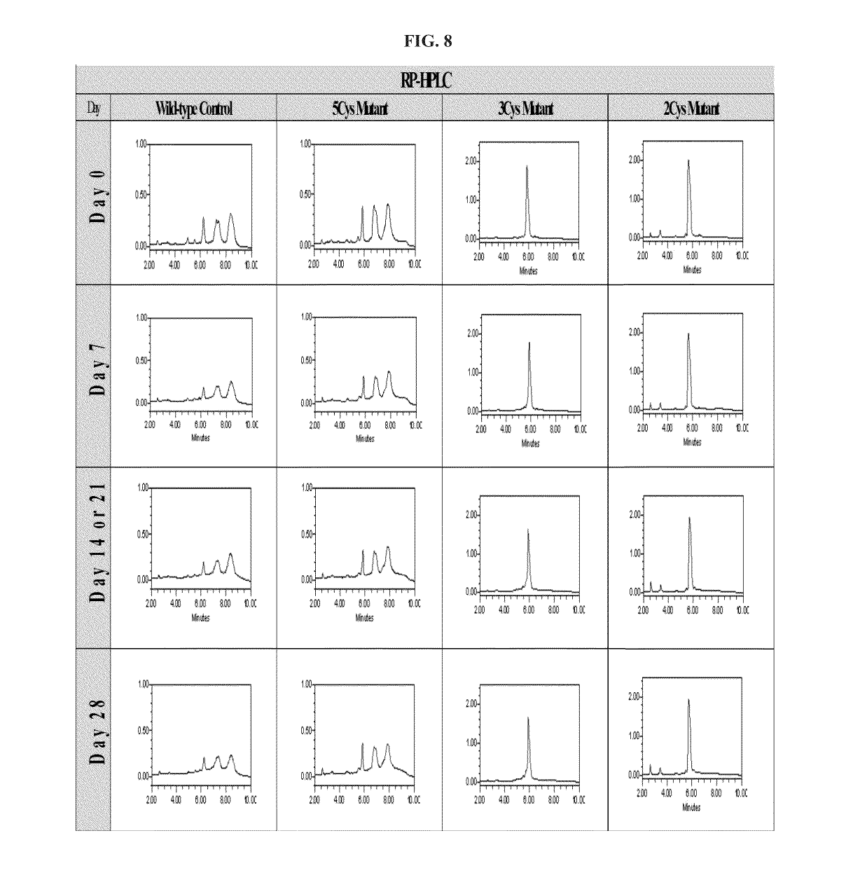

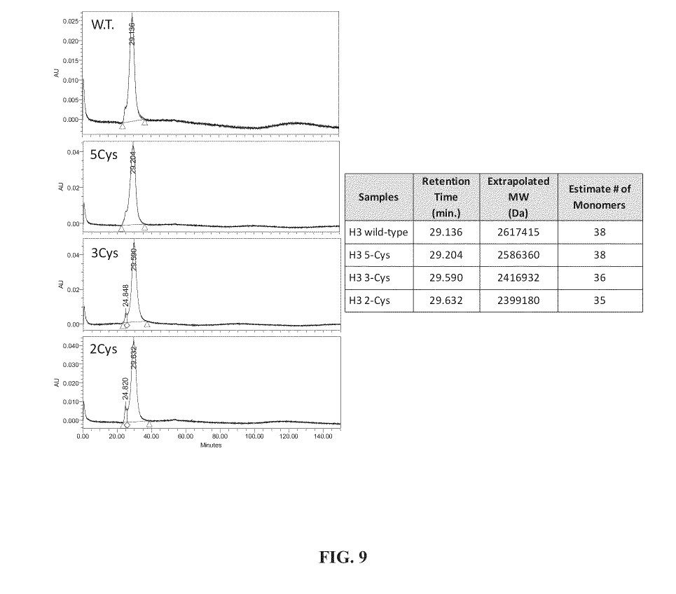

The RP-HPLC profiles for the 3Cys and 2Cys mutants are comparable but different from the wild-type and 5Cys mutant (FIG. 8). The 3Cys and 2Cys rHA are largely un-cross-linked and elute as a single peak while the wild-type and 5Cys rHA elute in multiple peaks due to various cross-linked populations of protein. Populations of cross-linked rHA are retained on the column due to increased hydrophobicity and elute later.

SRID-BCA Ratio—The 3Cys and 2Cys mutants have a higher SRID/BCA ratio than the wild-type and 5Cys mutant. The higher ratio for the 3Cys and 2Cys H3 rHA proteins may reflect a change in the antibody affinity or the reduced cross-linking in these mutants.

TABLE 19

| | SRID | BCA | |
|---|---|---|---|
| rHA Protein | μg/mL | μg/mL | SRID/BCA |
| H3 Perth wild-type | 400.5 | 465.0 | 0.86 |
| H3 Perth 5Cys | 456.7 | 497.3 | 0.92 |
| H3 Perth 3Cys | 649.1 | 474.4 | 1.37 |
| H3 Perth 2Cys | 770.3 | 503.9 | 1.53 |

Additional Testing—Dynamic light scattering (DLS), size exclusion chromatography (SEC), and electron microscopy (EM) assays were not included in the protocol but were performed in order to characterize the particle size of the H3 rHA proteins. Differential Scanning Fluorimetry (DSF) was also performed to compare thermal stability of the H3 rHA proteins, and the hemagglutination inhibition (HI) assay was performed to compare the antigenicity of the H3 rHA proteins.

DLS—The particle size of the rHA proteins by DLS is in the range characteristic of a rosette structures, 30-50 nm. The approximate transition temperatures by DLS are very similar for all H3 rHA proteins, 57-59° C.

TABLE 20

| | 25° C. | 45° C. | Estimated | |
|---|---|---|---|---|
| | Volume Mean | Volume Mean | Tm (° C.) | |
| | (d · nm) | (d · nm) | (Z-Average) | |
| H3 rHA | Average | Average | day 0 | day 28 |
| H3 Wild-type | 39 | 39 | 58.5 | 58.5 |
| 5Cys Mutant | 50 | 53 | 57.0 | 58 |
| 3Cys Mutant | 33 | 35 | 58.5 | 57.5 |
| 2Cys Mutant | 36 | 36 | 57.5 | 57.5 |

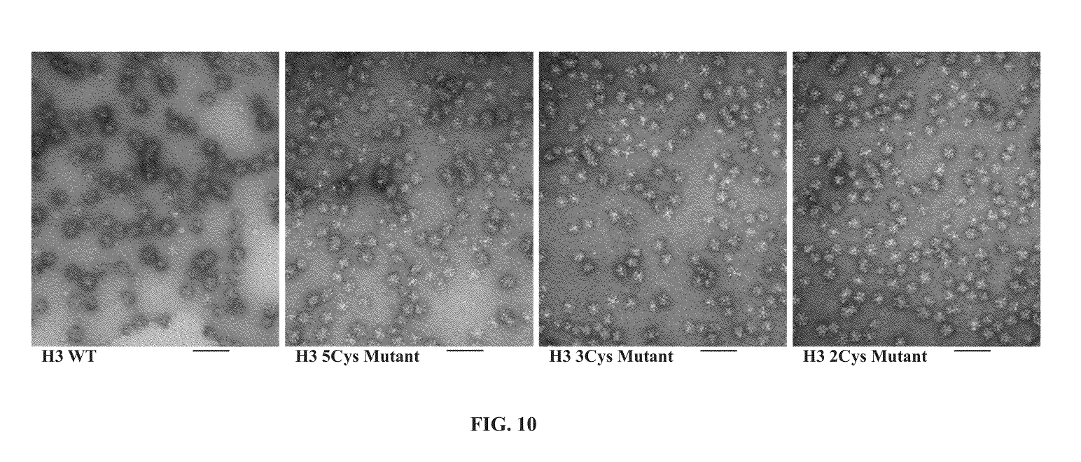

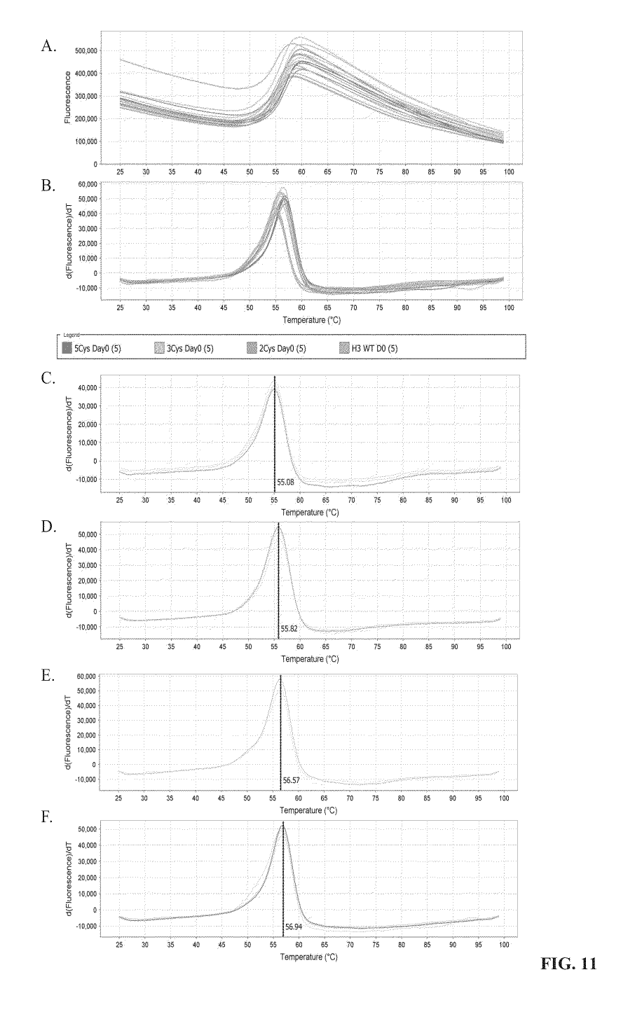

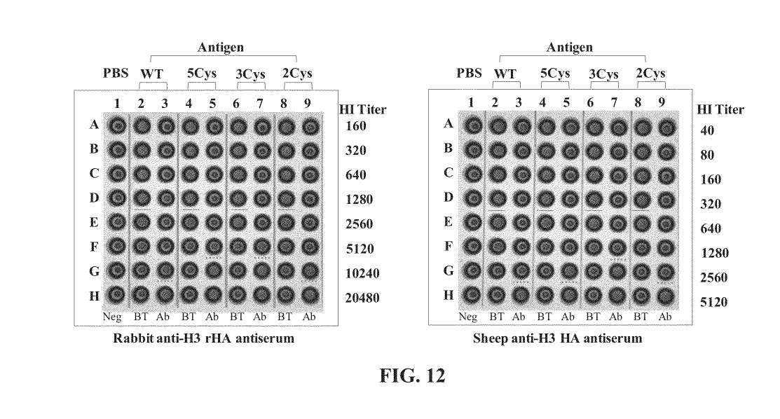

SEC—By SEC, the H3 rHA protein elute at essentially the same retention time. Extrapolated molecular weights in the range of 2.4-2.6 MDa were observed for the H3 rHA proteins. Using an approximate MW for the monomer of −70 kDa, the number of monomers per particle/rosette is estimated to be 35-38 (see FIG. 9).

EM—Electron microscopy was performed on the wild-type and cysteine mutant H3 rHA proteins. The wild-type and mutant rHA proteins form multimeric rosette-like structures approximately 30-40 nm in size. Under the same magnification and using the same protein concentration in the EM analysis, the density of rosette particles appears to be qualitatively similar among samples. Based on the analysis, higher order structure is unaffected by the cysteine mutagenesis.

DSF—H3/Perth rHA Wild-Type and cysteine mutants (2Cys, 3Cys, and 5Cys) were analyzed with Differential Scanning Fluorometry (DSF) in the presence of a molecular rotor dye (ProteoStat, Enzo Life Scienes) from 25° C. to 99° C. Fluorescence was monitored as a function of temperature and a single, large cooperative unfolding event was observed for each protein. The data show that all the H3/Perth rHA cysteine mutants had slightly greater thermal stability than wild-type H3 rHA, supporting the claim that mutating cysteine residues in the trasmembrane and/or cytoplasmic region of rHA proteins can enhance their stability.

TABLE 21
Melting Temperatures for H3 rHA Wild-type and Cys Mutants using DSF

| | | Standard |
|---|---|---|
| Protein | TM Mean (n = 5) | Deviation |
| H3 Perth rHA Wild-type | 55.08 | 0 |
| H3 Perth rHA 2Cys | 55.82 | 0 |
| H3 Perth rHA 3 Cys | 56.27 | 0.17 |
| H3 Perth rHA 5Cys | 56.71 | 0.20 |

The H3 rHA wild-type and cysteine mutant proteins were characterized in an antigenicity study using the hemagglutination inhibition (HI) test. The objective was to identify differences in the ability of the rHA proteins to bind specifically with antisera directed toward the H3 antigen. The H3 rHAs were standardized to have a hemagglutination titer of 4 HA units/25 μL, which results in agglutination in the first four wells of the back titration (BT) in the assay. The standardized quantity of each rHA was mixed with serially diluted antisera and the red blood cells added to determine the specific antibody binding of the antibody to the rHA molecule. Antisera produced in sheep against purified HA from H3 A/Wisconsin/15/2009-X-183 virus and antisera produced in rabbits using the wild-type H3 A/Perth 16/2009 rHA protein were used to evaluate the wild-type and mutant H3 A/Perth/16/2009 rHA proteins. The H1 titers obtained using the cysteine mutant rHAs were equivalent to or within 2-fold of the H1 titers obtained using the wild-type H3 rHA in assays with either the sheep or rabbit antisera. The results support a similar presentation of the antigenic sites on the wild-type and mutant H3 rHA proteins.

### Example 4

### Mechanism of Potency Loss

This Example was established to determine the mechanism of potency loss using an H3 rHA protein as a model system. A real time stability study was performed using freshly purified H3 A/Victoria/361/2011 (H3 Victoria) rHA.

A 28 day stability study was performed using three formulations of the H3 A/Victoria/361/2011 (H3 Victoria) rHA protein, and two different storage temperatures.

TABLE 22
Formulations and Storage Conditions for H3 Victoria rHA in Stage II.

| | | Storage |
|---|---|---|
| Sample | Formulation | Conditions |
| Pre-formulated Retentate | Standard* | 2-8° C. |
| Pre-formulated Retentate | Standard* | 22-28° C. |
| STG-Citrate** | Standard*, +70 mM STG, | 2-8° C. |
| | 34 mM Citrate | |
| Monovalent Bulk | Standard*, +0.04% Triton X-100 | 2-8° C. |

*Standard Formulation: 10 mM Sodium Phosphate, 150 mM Sodium Chloride, 0.005% Tween-20, pH 6.8-7.2.
**Reference is made to Examples 5 and 6.

The formulations were evaluated in the SRID assay for potency, for free thiol content using a fluorescence based assay, and for free Cys using peptide mapping.

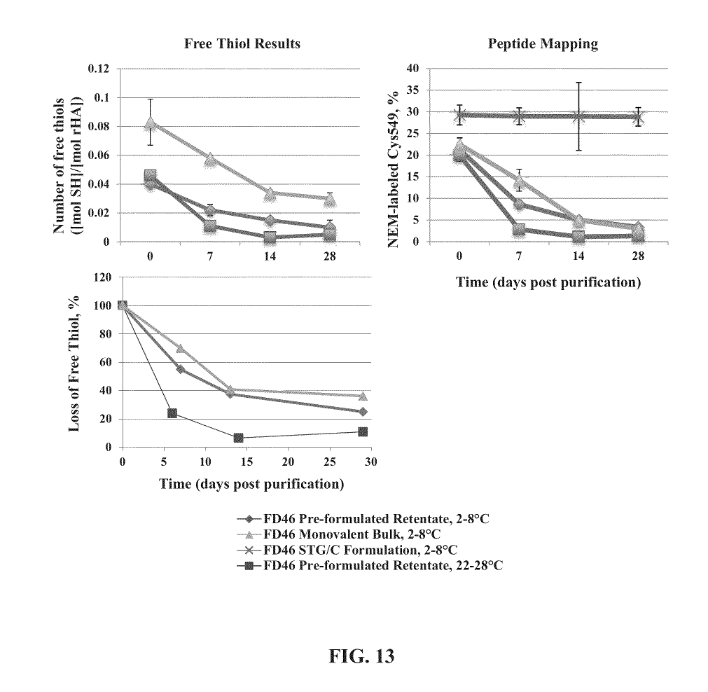

The free thiol content diminishes in the Pre-formulated Retentate stored at 2-8° C. and 22-28° C., and in the Monovalent Bulk stored at 2-8° C. (FIG. 13). The assay could not be performed with the STG-Citrate formulation due to interference from the STG in the formulation. Peptide mapping shows a loss of free cysteine (NEM-labeled cysteine) at position 549 in the same formulations in agreement with the free thiol results. In contrast, the level of free cysteine at position 549 remains the same in the STG-Citrate formulation, the most stable formulation.

The number of free thiols is small, less than one per molecule of rHA on day 0; however, the relative loss in free thiol content over the course of the study is large. At the end of the study, the total initial free thiol content is reduced by 90% in the Pre-formulated Retentate stored at 22-28° C. and by approximately 70% in the Pre-formulated Retentate and the Monovalent Bulk samples stored at 2-8° C. Similarly, the total available free cysteine at position 549, approximately 20% for the Pre-formulated Retentate and Monovalent Bulk samples, is almost completely depleted in these formulations (<5%) by the end of the study. In contrast, the starting level of free Cys549 is greater in the STG-Citrate formulation (−30%), and does not change during storage.

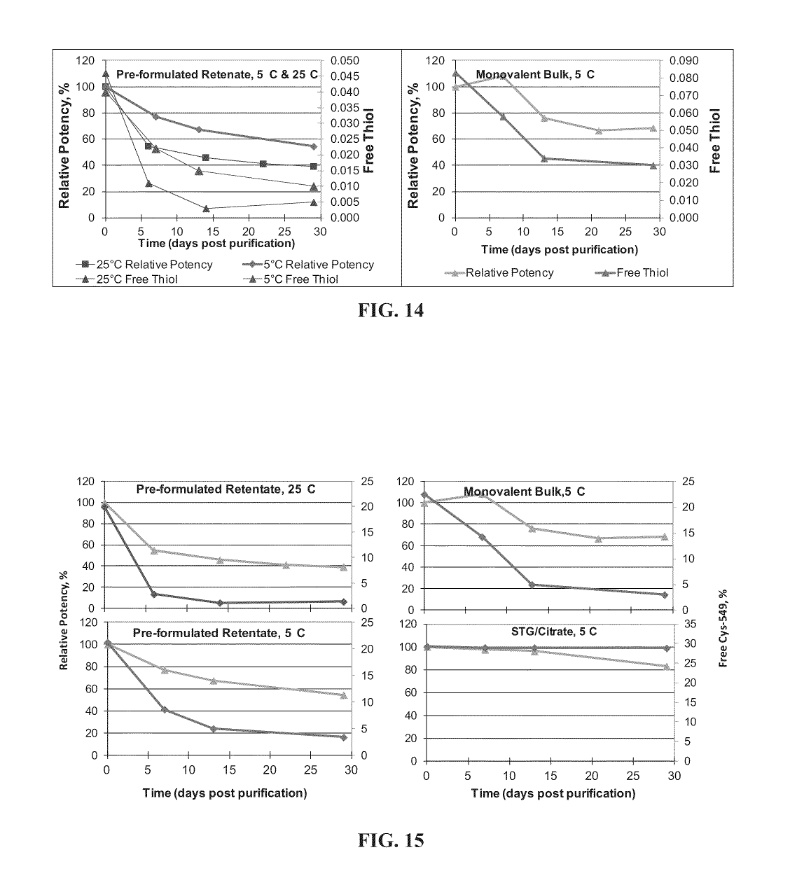

The results for free thiol and the loss of Cys549 from peptide mapping correlate with the loss of potency for all formulations in the study. The rate of potency loss, and rates of free thiol loss and Cys549 loss is greatest for the Pre-formulated Retentate stored at 22-28° C. followed by the Pre-formulated Retentate and Monovalent Bulk samples stored at 2-8° C. The relative potency values for the formulations are plotted alongside the relative change in free thiol content in FIG. 14 and alongside the relative change in free Cys549 in FIG. 15.

### Example 5

### Formulations Containing Citrate and STG

This Example was designed to focus on the promising formulations, those containing citrate and sodium thioglycolate (STG). The objective was to identify an optimal citrate concentration for formulations with a small concentration of STG and to determine whether citrate or STG alone could improve the stability of the formulation. The rHA used in this study was obtained from a process validation lot using B/Brisbane (45-09018), H1/Brisbane (45-09012) and H3/Brisbane (45-09023 and 45-09025). This lot was filled at Hospira One-2-One in McPherson, K S, and is referred to as “PV2” or as the Hospira number, “CMO-119.” Using aseptic technique (hood HD 016), 400 vials of CMO-119 drug product were pooled into a sterile bottle. This pooled material was subdivided and modified by addition of concentrated excipient to yield the desired formulations (Table 23).

TABLE 23
Formulations

| | | Excipient (mg/mL) | | |
|---|---|---|---|---|
| | | Citrate | STG | |
| 1 | Air Ctrl | 0 | 0 | Control - current formulation |
| 2 | N₂ Ctrl | 0 | 0 | N₂ Control - All samples other than 1 |
| | | | | w/ N₂ overlay |
| 3 | C20 | 20 | 0 | 20 mg/mL Citrate no STG - compare |
| | | | | to #7 |
| 4 | S | 0 | 0.2 | STG control - no citrate |
| 5 | C5S | 5 | 0.2 | Citrate series w/ reducing agent STG |
| 6 | C10S | 10 | 0.2 | |
| 7 | C20S | 20 | 0.2 | |

Samples were set at 35° C., 25° C., and 5° C., and scheduled for pulls normally set at intervals of 1 week. An additional 2-day pull was scheduled for the 35° C. samples, fewer early time points were scheduled for the 5° C. samples, and reserve samples were set for long time points, if warranted. The focus of the SRID potency measurements was H1/Brisbane rHA, but frequent measurements were also made for H3/Brisbane and B/Brisbane.

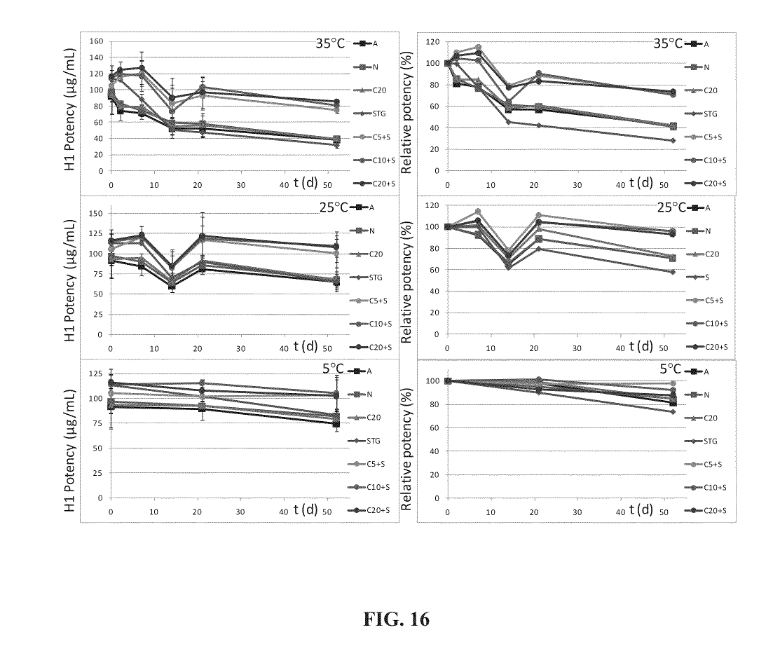

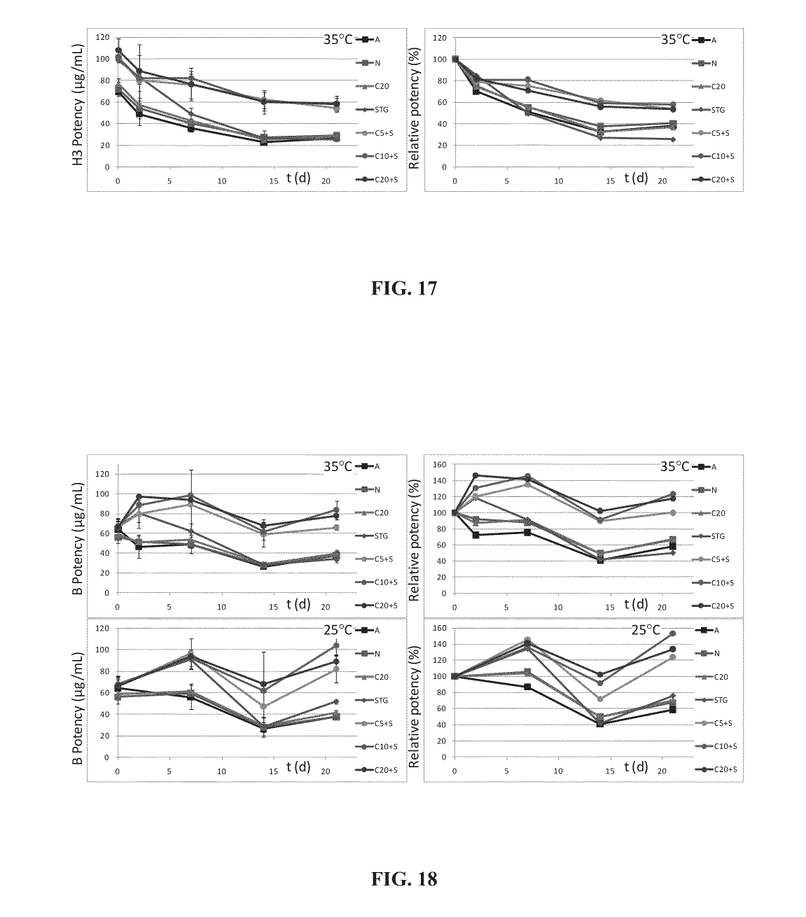

SRID potency measurements are listed in Tables 23-25, and these data are plotted in FIGS. 16-18.

TABLE 24
Hi/Brisbane SRID Potency.

| | H1 | | | | | | | | | | | | | |
|---|---|---|---|---|---|---|---|---|---|---|---|---|---|---|
| | 35° C. | | | | | | 25° C. | | | | | 5° C. | | |
| | Day | | | | | | | | | | | | | |
| | 0 | 2 | 7 | 14 | 21 | 52 | 0 | 7 | 14 | 21 | 52 | 0 | 21 | 52 |
| A | 91.4 | 73.9 | 71.3 | 52.1 | 52.0 | 38.3 | 91.4 | 84.4 | 59.7 | 81.1 | 64.9 | 91.4 | 89.5 | 74.5 |
| N | 96.9 | 83.0 | 74.4 | 59.3 | 57.8 | 39.6 | 96.9 | 90.0 | 64.1 | 85.6 | 68.2 | 96.9 | 92.4 | 82.0 |
| C20 | 93.3 | 79.0 | 79.1 | 54.6 | 56.6 | 39.4 | 93.3 | 95.0 | 66.2 | 91.5 | 67.7 | 93.3 | 92.6 | 79.3 |
| STG | 113.1 | 112.4 | 88.4 | 51.0 | 47.6 | 31.7 | 113.1 | 113.1 | 69.7 | 89.6 | 65.1 | 113.1 | 101.8 | 83.0 |
| C5 + S | 105.2 | 115.8 | 120.8 | 83.5 | 93.1 | 75.3 | 105.2 | 120.1 | 82.2 | 116.7 | 100.5 | 105.2 | 102.2 | 103.2 |
| C10 + S | 114.2 | 119.5 | 117.0 | 73.5 | 103.3 | 80.6 | 114.2 | 120.5 | 83.6 | 118.5 | 109.5 | 114.2 | 115.3 | 105.2 |
| C20 + S | 116.4 | 124.3 | 127.2 | 90.2 | 96.9 | 85.6 | 116.4 | 123.1 | 85.1 | 121.6 | 108.2 | 116.4 | 107.7 | 102.4 |

TABLE 25
H3/Brisbane SRID Potency

| | H3 | | | | | | | | |
|---|---|---|---|---|---|---|---|---|---|
| | 35° C. | | | | | 25° C. | | 5° C. | |
| | Day | | | | | | | | |
| | 0 | 2 | 7 | 14 | 21 | 0 | 14 | 0 | 21 |
| A | 69.8 | 48.9 | 35.6 | 22.6 | 26.7 | 69.8 | 36.9 | 69.8 | 49.5 |
| N | 72.2 | 54.1 | 39.9 | 27.2 | 29.2 | 72.2 | 37.9 | 72.2 | 46.5 |
| C20 | 76.9 | 57.4 | 42.9 | 25.0 | 28.2 | 76.9 | 41.4 | 76.9 | 52.5 |
| STG | 98.5 | 83.6 | 48.9 | 26.8 | 25.2 | 98.5 | 64.5 | 98.5 | 71.5 |
| C5 + S | 101.5 | 80.0 | 76.1 | 62.4 | 54.1 | 101.5 | 78.5 | 101.5 | 70.0 |
| C10 + S | 101.5 | 82.2 | 82.2 | 60.0 | 58.6 | 101.5 | 88.3 | 101.5 | 82.3 |
| C20 + S | 108.1 | 88.9 | 76.6 | 60.3 | 57.9 | 108.1 | 84.9 | 108.1 | 74.3 |

TABLE 26
B/Brisbane SRID Potency

| | B | | | | | | | | | | |
|---|---|---|---|---|---|---|---|---|---|---|---|
| | 35° C. | | | | | 25° C. | | | | 5° C. | |
| | Day | | | | | | | | | | |
| | 0 | 2 | 7 | 14 | 21 | 0 | 7 | 14 | 21 | 0 | 21 |
| A | 64.4 | 46.4 | 48.6 | 26.4 | 37.6 | 64.4 | 55.8 | 26.4 | 37.8 | 64.4 | 49.3 |
| N | 56.1 | 51.3 | 49.2 | 27.8 | 37.4 | 56.1 | 59.2 | 27.8 | 37.8 | 56.1 | 46.3 |
| C20 | 58.9 | 51.2 | 53.5 | 29.1 | 39.8 | 58.9 | 61.1 | 29.1 | 41.5 | 58.9 | 52.3 |
| STG | 68.0 | 80.2 | 62.0 | 28.3 | 33.9 | 68.0 | 91.0 | 28.3 | 51.7 | 68.0 | 71.7 |
| C5 + S | 66.0 | 79.4 | 88.9 | 59.1 | 65.9 | 66.0 | 96.2 | 47.3 | 81.8 | 66.0 | 69.7 |
| C10 + S | 67.7 | 88.7 | 98.6 | 61.8 | 83.6 | 67.7 | 92.1 | 61.8 | 103.7 | 67.7 | 82.0 |
| C20 + S | 66.4 | 97.1 | 93.8 | 67.8 | 77.8 | 66.4 | 93.7 | 67.8 | 88.8 | 66.4 | 74.0 |

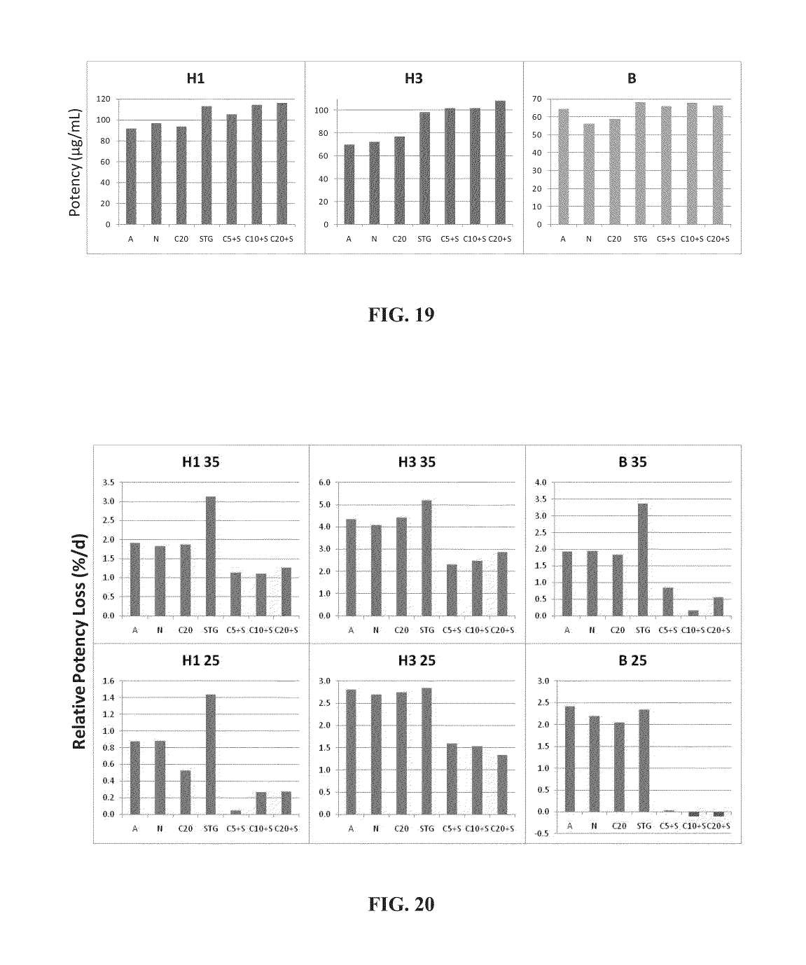

At t=0, the measured potency of these formulations indicated that the excipients did not affect the apparent potency as measured by SRID for B/Brisbane. There was a small effect of the excipient for H1/Brisbane and a moderate effect for H1/Brisbane (FIG. 19). Based on the fact that the potency measured for samples with STG alone were equivalent to those with STG and citrate, the effect appears to be due to the presence of a reducing agent. It is not yet known whether the reducing agent affects the assay directly or alters the conformation of the rHA so as to better match the SRID reagents.

For all H1, H3, and B, the stability was improved by citrate and STG, but not by either of the excipients individually. Formulations with STG alone exhibited the poorest stability; the slope of stability curves (relative potency as a function of time) was over 60% higher for three of the storage conditions (FIG. 20). The slopes for citrate-containing samples with STG did not show a consistent concentration dependence, but all reflected a significant improvement in the stability of the formulation. The ratio of the control (A) slope to the mean slope of citrate+STG formulations to was at least 1.6 and as high as 4.4.

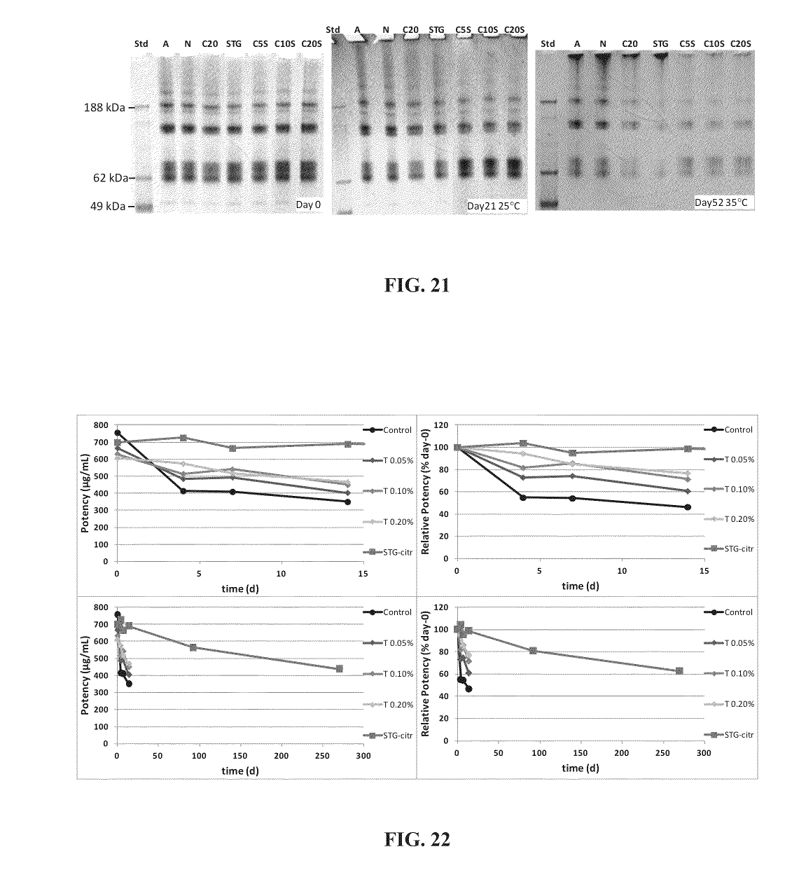

SDS-PAGE results are shown in FIG. 21. Day-0 data show that all formulations are initially equivalent. By day-21, it is clear that there is less aggregation in formulations containing both STG and citrate. At the end of study (day-52), some aggregate has become visible in formulations containing both STG and citrate, but the predominant bands are HA0. It is not clear why the overall intensity appears lower, but the SRID potency for H1 was still approximately 80% of the day-0 value. In these measurements, as in previous studies, the loss of SRID potency correlates with the accumulation of aggregate observed in nonreducing SDS-PAGE.

This Example was designed to for monitoring the potency of each rHA in trivalent formulations containing lead excipients. Particle size (by DLS) and aggregation (by non-reducing SDS-PAGE) were also measured, although these parameters are averages over all rHAs and are not specific to rHA from a specific strain. The excipients tested were citrate and sodium thioglycolate. In order to minimize internal disulfide reduction, STG was used at a very low concentration (0.2 mg/mL). The overall conclusions are:

Stability was improved for all three rHAs (H1/Brisbane, H3/Brisbane, and B/Brisbane) in formulations with both citrate and STG. Citrate alone (20 mg/mL) does not improve stability. STG alone has a negative effect on stability. The highest concentration tested (5 mg/mL) adversely affects stability (data not shown). Lower concentrations may or may not be effective.

In the presence of both citrate and STG, aggregation of rHA was minimal. The degree of aggregation did not decrease below that observed on day-0.

### Example 6

### Early-Phase Stability Study for H3 Perth with 0.035% Triton X-100

This Example was designed to (a) evaluate the stability of H3 Perth formulated in manufacturing with 0.035% Triton X-100 and (b) to better understand the unexpectedly high stability of a lot of H3/Wisconsin in stability testing, and (c) to compare the stability of an STG-citrate formulation to the formulations with high concentrations of Triton X-100. Retrospective testing showed that this lot had an unusually high Triton X-100 concentration of approximately 0.2%. In this study, H3 Perth was formulated in Manufacturing to a Triton X-100 concentration of 0.035%. This lot was supplemented with Triton X-100 to simulate the concentration used in formulation development studies, 0.05%, and to concentrations designed to test the hypothesis that the observed enhanced stability of H3/Wisconsin was due to elevated Triton X-100 (0.1%, 0.2%). Another formulation was prepared in which the lot was supplemented with 1% sodium citrate and 0.02% sodium thioglycolate.

The formulations tested are listed in Table 27. Because the Triton X-100 stock was added following the initial formulation, some dilution occurred, but was only 1.6% at the highest Triton concentration. Dilution of the STG-citrate formulation was 9.4%. All samples were stored at 25° C.

TABLE 27
Formulations

| | H3 | Triton X-100 | | | | Dilution |
|---|---|---|---|---|---|---|
| | (mL) | final | to add | Citrate | STG | (%) |
| C | 5.0 | 0.035% | 0 | 0 | 0 | 0 |
| T05 | 5.0 | 0.05% | 0.015% | 0 | 0 | 0.15 |
| T10 | 5.0 | 0.10% | 0.065% | 0 | 0 | 0.65 |
| T20 | 5.0 | 0.20% | 0.165% | 0 | 0 | 1.65 |
| STG-Citr | 5.0 | 0.035% | 0 | 1% | 0.02% | 9.4 |

The SRID results for samples stored under accelerated conditions are listed in Table 6 and plotted in FIG. 22. These results show that for the control samples (0.35% Triton X-100), the potency drops quickly to approximately one half the day-0 potency. At higher levels of Triton X-100, the potency decreases more slowly and does not decrease as much. The stability is better in the presence of 0.1% or 0.2% Triton X-100 than with 0.05% Triton X-100, but the difference between 0.1% and 0.2% Triton X-100 is negligible. The potency of the STG-citrate formulation changed very little over the two-week accelerated stability period and maintained over 80% of the original potency for 92 days.

TABLE 28
Potency according to SRID - All data are listed as μg/mL.

| | day 0 | day 4 | day 7 | day 14 | day 92 | day 270 |
|---|---|---|---|---|---|---|
| Control | 755 | 413 | 409 | 349 | — | — |
| 0.05% | 665 | 485 | 494 | 403 | — | — |
| 0.10% | 630 | 514 | 540 | 449 | — | — |
| 0.20% | 607 | 573 | 515 | 466 | — | — |
| STG-Citr | 698 | 726 | 664 | 689 | 563 | 487 |

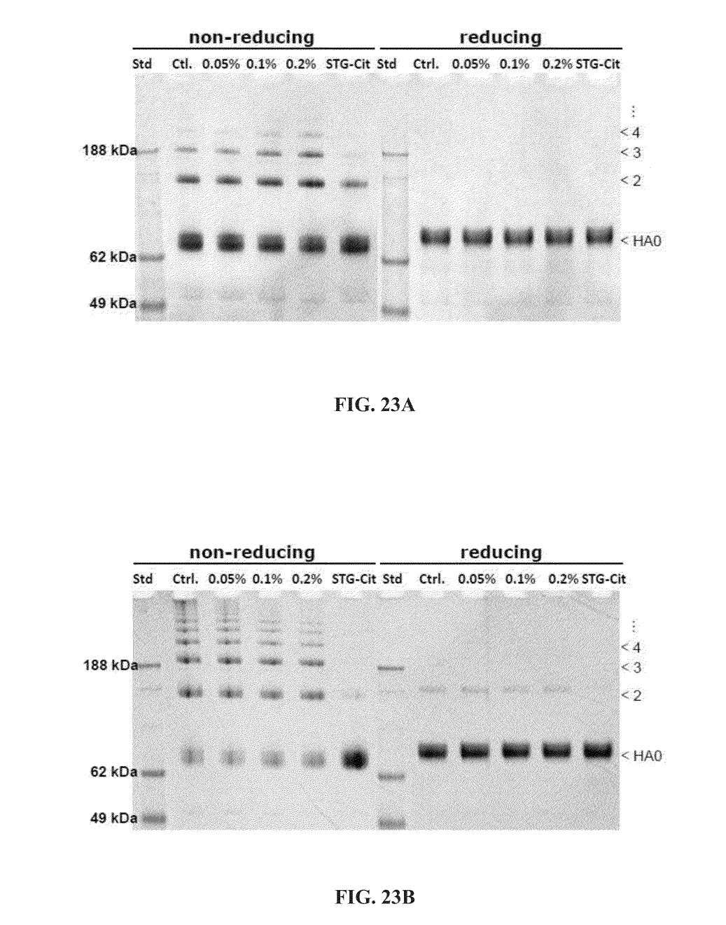

SDS-PAGE results are shown in FIGS. 23A-B. The initial pattern shows that most of the rHA was in the form of monomer (HA0), with some cross-linked dimer and trimer present. The protein appears to be cross-linked by disulfide bonds, as reducing gels indicate that essentially all of the protein is HA0. Within two weeks at 25° C., the amount of monomeric rHA has decreased significantly and some of the cross-linked dimer is non-reducible. The formulations with higher concentrations of Triton X-100 have less cross-linking than the control (0.035% Triton X-100). Disulfide cross linking in the formulation with citrate and STG showed little change over two weeks and showed no evidence of non-reducible cross-links.

The data in FIG. 23 shows that Triton X-100 improves the stability of H3 Perth rHA, but 0.035% Triton X-100 does not provide as much improvement as 0.05%. At 0.1%, Triton X-100 further improves stability and further increasing to 0.2%, provides an incremental improvement to stability. This was unexpected, as previous results had shown that formulations with 0.05, 0.08, or 0.15% Triton X-100 had similar stability.

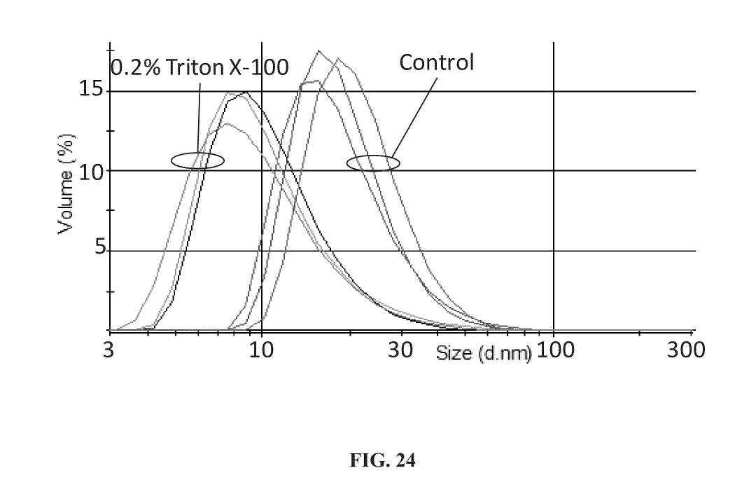

Day-0 DLS results showed that increasing Triton X-100 concentrations resulted in decreased average particle size. FIG. 24 shows that there is minimal difference over the course of the 14 day study, but the presence of a high concentration of Triton X-100 significantly decreased the average particle size.

### Example 7

### Immunogenicity of rHA is not Affected by the STG-Citrate Formulation

This Example was designed to evaluate the effect of the STG citrate formulation on the immunogenicity of rHA. Using H1 California/07/2009, two formulations were prepared at an rHA concentration of 120 μg/mL. The control formulation was in the formulation buffer used in Flublok (10 mM sodium phosphate, 150 mM sodium chloride, 0.005% Tween-20, pH6.8-7.2). The second formulation was identical except that 0.02% sodium thioglycolate (STG) and 1% sodium citrate were added to the formulation. These formulations were administered intramuscularly to 6-8 week old Balb/c mice in two doses: 3 μg and 0.3 μg. The 3 μg dose was administered as a 25 μL dose of each formulation and the 0.3 μg dose was administered as a 25 μL dose of a 1:10 dilution of each formulation. Mice were dosed on day-0 and on day-21. Eight mice were used in each of the four cohorts: High Dose Control, High Dose STG, Low Dose Control, and Low Dose STG. Blood samples were taken prior to dosing on day-0, on day-21, and on day-42. Blood samples were allowed to clot and then centrifuged, and the resulting serum stored at −20° C. Serum samples were tested for antibody titer using hemagglutination inhibition (HAI) and ELISA.

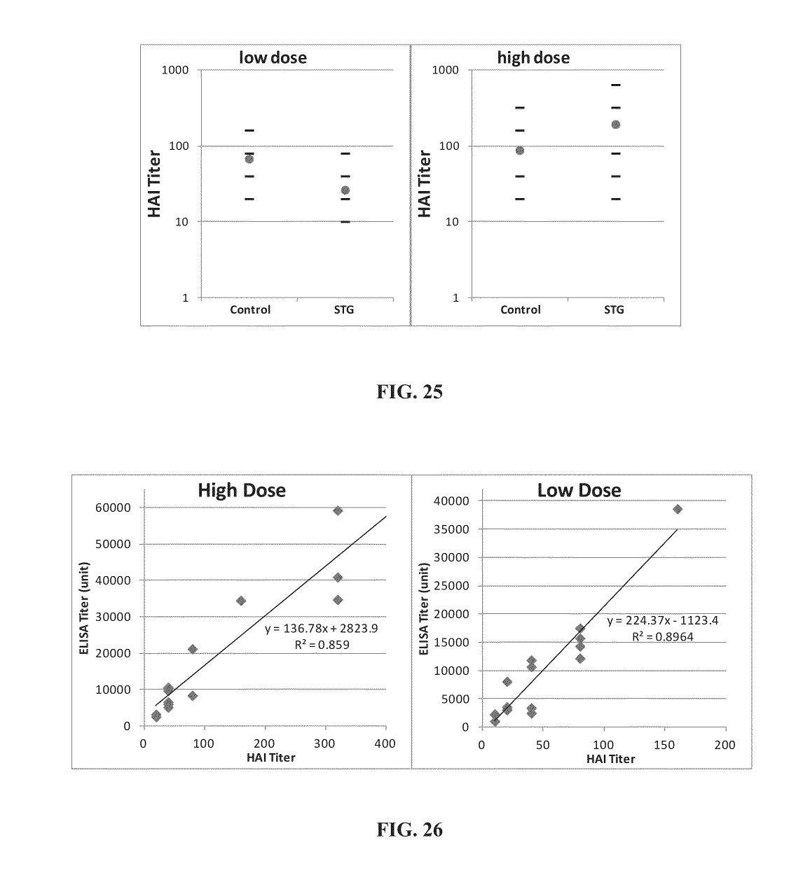

The HAI titers are shown in Table 29 and FIG. 25. These results show that the STG-citrate formulation does not have a significant effect on immunogenicity of H1 California rHA.

TABLE 29
HAI titers - Titers are listed as the reciprocals of the
highest dilutions for which there was no agglutination.

| | HAI Titers (day-42) | | | | | | | | |
|---|---|---|---|---|---|---|---|---|---|
| | m1 | m2 | m3 | m4 | m5 | m6 | m7 | m8 | Mean |
| Ctrl low dose | 80 | 80 | 20 | 40 | 80 | 40 | 160 | 40 | 67 |
| STG low dose | 10 | 20 | 10 | 80 | 40 | 20 | 20 | 10 | 26 |
| Ctrl high dose | 40 | 40 | 160 | 40 | 40 | 20 | 40 | 320 | 88 |
| STG high dose | 40 | 40 | 640 | 320 | 320 | 80 | 20 | 80 | 193 |

The ELISA titers determined for serum from day-42 are shown in Table 30. These values were calculated by normalizing data for each mouse to the day-0 (non-immunized) ELISA response. These results show that the ELISA titers for the STG and Control formulations are not significantly different. FIG. 26 shows that the ELISA and HAI results are proportionate. Titers obtained using the two methods are plotted as a scatter plot. The ELISA and HAI results demonstrate that the STG-citrate formulation does not affect the immunogenicity of rHA.

TABLE 30
ELISA titers normalized to a day-0 baseline.

| | ELISA Titers (day-42) | | | | | | | | |
|---|---|---|---|---|---|---|---|---|---|
| | m1 | m2 | m3 | m4 | m5 | m6 | m7 | m8 | Mean |
| Ctrl low dose | 15717 | 12183 | 3069 | 3421 | 17505 | 11856 | 38605 | 2506 | 13108 |
| STG low dose | 1073 | 8089 | 2229 | 14325 | 10685 | 3578 | 3147 | 2320 | 5681 |
| Ctrl high dose | 10585 | 5112 | 34496 | 10343 | 6555 | 3185 | 5989 | 40903 | 14646 |
| STG high dose | 9691 | NA | 6600 | 34725 | 59291 | 8348 | 2516 | 21180 | 17794 |

The invention is further described by the following numbered paragraphs:

1. An isolated, non-naturally occurring recombinant hemagglutinin (rHA) protein comprising one or more cysteine mutations.

2. The protein of paragraph 1, wherein the rHA protein is a H1 protein.

3. The protein of paragraph 1, wherein the H1 protein is isolated from a California or Solomon strain.

4. The protein of paragraph 3, wherein the California strain is a California/07/2009 strain.

5. The protein of paragraph 3, wherein the Solomon strain is a Solomon Is/03/2006 strain.

6. The protein of any one of paragraphs 2-5, wherein the cysteine mutation is in the carboxy terminus region.

7. The protein of any one of paragraphs 2-6, wherein the cysteine mutation is in the transmembrane region or cytosolic region.

8. The protein of paragraph 1, wherein the rHA protein is a B protein.

9. The protein of paragraph 8, wherein the B protein is isolated from a Brisbane, Florida, Ohio, Jiangsu or Hong Kong strain.

10. The protein of paragraph 9, wherein the Brisbane strain is a Brisbane/60/2008 strain.

11. The protein of paragraph 9, wherein the Florida strain is a Florida/04/2006 strain.

12. The protein of paragraph 9, wherein the Ohio strain is a Ohio/01/2005 strain.

13. The protein of paragraph 9, wherein the Jiangsu strain is a Jiangsu/10/2003 strain.

14. The protein of paragraph 9, wherein the Hong Kong strain is a Hong Kong/330/2001 strain.

15. The protein of any one of claims 8-14, wherein the cysteine mutation is in the carboxy terminus region which includes the transmembrane (TM) and cytosolic (CT) domains.

16. The protein of paragraph 1, wherein the rHA protein is a H3 protein.

17. The protein of paragraph 16, wherein the H3 protein is isolated from a Victoria, Perth, Brisbane or Wisconsin strain.

18. The protein of paragraph 17, wherein the Victoria strain is a Victoria/361/2011 strain.

19. The protein of paragraph 17, wherein the Perth strain is a Perth/16/2009 strain.

20. The protein of paragraph 19, wherein the mutation is C524S and/or C528A.

21. The protein of paragraph 19, wherein the mutation is C524A, C528A, C539A, C546A and/or C549A.

22. The protein of paragraph 19, wherein the mutation is C539A, C546A and/or C549A.

23. The protein of paragraph 17, wherein the Brisbane strain is a Brisbane/16/2007 strain.

24. The protein of paragraph 17, wherein the Wisconsin strain is a A/Wisconsin/67/05 strain.

25. The protein of any one of paragraphs 16-24, wherein the cysteine mutation is in the transmembrane region.

26. The protein of any one of paragraphs 16-24, wherein the cysteine mutation is in the carboxy terminus region.

27. A baculovirus vector encoding and expressing a nucleotide sequence expressing any one of the proteins of paragraphs 1-26.

28. An influenza vaccine comprising any one of the proteins of paragraphs 1-26.

29. An influenza vaccine comprising the baculovirus vector of paragraph 27.

30. A method for stabilizing a rHA protein comprising identifying one or more cysteine residues in the rHA protein, mutating the one or more cysteine residues to an amino acid residue that is not cysteine and does not disrupt trimer formation, thereby stabilizing the rHA protein.

31. The method of paragraph 30, wherein the protein is any one of the proteins of paragraphs 1-26.

32. A stabilized protein formulation comprising (a) a protein, (b) a citrate and (c) a thioglycolate or a thioglycerol.

33. A method for stabilizing a protein formulation comprising adding a citrate and a thioglycolate or a thioglycerol to the formulation.

34. The formulation or method of paragraph 32 or 33, wherein the thioglycolate is sodium thioglycolate.

35. The formulation or method of paragraph 32 or 33, wherein the thioglycerol is monothioglycerol.

36. The formulation or method of any one of paragraphs 32-35, wherein the concentration of the citrate is at least about 1 mg/ml.

37. The formulation or method of any one of paragraphs 32-36, wherein the concentration of the citrate is at least about 5 mg/ml.

38. The formulation or method of any one of paragraphs 32-37, wherein the concentration of the citrate is at least about 10 mg/ml.

39. The formulation or method of any one of paragraphs 32-38, wherein the concentration of the thioglycolate or thioglycerol is about 0.2 mg/ml.

40. The formulation or method of any one of paragraphs 32-39, wherein the formulation is a vaccine.

41. The formulation or method of paragraph 40, wherein the vaccine is an influenza vaccine.

42. The formulation or method of paragraph 41, wherein the influenza vaccine is a trivalent vaccine.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An isolated, non-naturally occurring recombinant hemagglutinin (rHA) protein comprising one or more cysteine mutations.

2. The protein of claim 1, wherein the rHA protein is a H1, H2, H3, H5, H7 or H9 protein.

3. The protein of claim 2, wherein the cysteine mutation is in the carboxy terminus region.

4. The protein of claim 2, wherein the cysteine mutation is in the transmembrane region.

5. The protein of claim 2, wherein the cysteine mutation is in the cytosolic region.

6. The protein of claim 1, wherein the rHA protein is a B protein.

7. The protein of claim 6, wherein the cysteine mutation is in the carboxy terminus region which includes the transmembrane (TM) and cytosolic (CT) domains.

8. A baculovirus vector encoding and expressing a nucleotide sequence expressing the protein of claim 1.

9. An influenza vaccine comprising the protein of claim 1.

10. An influenza vaccine comprising the baculovirus vector of claim 8.

11. A method for stabilizing a rHA protein comprising identifying one or more cysteine residues in the rHA protein, mutating the one or more cysteine residues to an amino acid residue that is not cysteine and does not disrupt trimer formation, thereby stabilizing the rHA protein.

12. The method of claim 11, wherein the rHA protein is a H1, H2, H3, H5, H7 or H9 protein.

13. The method of claim 12, wherein the cysteine mutation is in the carboxy terminus region.

14. The method of claim 12, wherein the cysteine mutation is in the transmembrane region.

15. The method of claim 12, wherein the cysteine mutation is in the cytosolic region.

16. The method of claim 11, wherein the rHA protein is a B protein.

17. The method of claim 16, wherein the cysteine mutation is in the carboxy terminus region.

18. A stabilized protein formulation comprising (a) a protein, (b) a citrate and (c) a thioglycolate or a thioglycerol.

19. A method for stabilizing a protein formulation comprising adding a citrate and a thioglycolate or a thioglycerol to the formulation.

20. The formulation or method of claim 18 or 19, wherein the thioglycolate is sodium thioglycolate.

21. The formulation or method of claim 18 or 19, wherein the thioglycerol is monothioglycerol.

22. The formulation or method of claim 18 or 19, wherein the concentration of the citrate is at least about 1 mg/ml.

23. The formulation or method of claim 18 or 19, wherein the concentration of the thioglycolate or thioglycerol is about 0.2 mg/ml.

24. The formulation or method of claim 18 or 19, wherein the formulation is a vaccine.

25. The formulation or method of claim 24, wherein the vaccine is an influenza vaccine.

26. The formulation or method of claim 25, wherein the influenza vaccine is a trivalent vaccine.


SEQUENCE LISTING (13 sequences)

SEQ ID NO: 1 - DNA, 44 bp, Artificial Sequence
  Description of Artificial Sequence Synthetic primer
cctttgccatatcagcttttttgcttgctgttgctttgttgggg

SEQ ID NO: 2 - DNA, 44 bp, Artificial Sequence
  Description of Artificial Sequence Synthetic primer
ccccaacaaagcaacagcaagcaaaaaagctgatatggcaaagg

SEQ ID NO: 3 - DNA, 68 bp, Artificial Sequence
  Description of Artificial Sequence Synthetic primer
ggggttcatcatgtgggccgcccaaaaaggcaacattagggccaacattgccatttaagtaagtaccg

SEQ ID NO: 4 - DNA, 68 bp, Artificial Sequence
  Description of Artificial Sequence Synthetic primer
cggtacttacttaaatggcaatgttggccctaatgttgcctttttgggcggcccacatgatgaacccc

SEQ ID NO: 5 - DNA, 44 bp, Artificial Sequence
  Description of Artificial Sequence Synthetic primer
cctttgccatatcatcttttttgcttgctgttgctttgttgggg

SEQ ID NO: 6 - DNA, 44 bp, Artificial Sequence
  Description of Artificial Sequence Synthetic primer
ccccaacaaagcaacagcaagcaaaaaagatgatatggcaaagg

SEQ ID NO: 7 - protein, 36 aa, Human influenza virus
ILWISFAISCFLLCVALLGFIMWACQKGNIRCNICI

SEQ ID NO: 8 - protein, 37 aa, Human influenza virus
ILAIYSTVASSLVLVVSLGAISFWMCSNGSLQCRICI

SEQ ID NO: 9 - protein, 37 aa, Human influenza virus
ILAIYSTVASSLVLLVSLGAISFWMCSNGSLQCRICI

SEQ ID NO: 10 - protein, 37 aa, Human influenza virus
ILLYYSTAASSLAVTLMIAIFVVYMVSRDNVSCSICL

SEQ ID NO: 11 - protein, 37 aa, Human influenza virus
ILLYYSTAASSLAVTLMLAIFIVYMVSRDNVSCSICL

SEQ ID NO: 12 - protein, 37 aa, Human influenza virus
ILLYYSTAASSLAVTLMIAIFIVYMVSRDNVSCSICL

SEQ ID NO: 13 - protein, 36 aa, Human influenza virus; identical to SEQ ID NO: 7